(19) United States
(12) Patent Application Publication
(10) Pub. No.: US 2026/0317405 A1
(43) Pub. Date: Oct. 15, 2026

(54) THERAPEUTIC AGENTS AND COMPOSITIONS AND RELATED METHODS

(71) Applicant: KORU BIOTECH SOLUTIONS LIMITED, Palmerston North (NZ)

(72) Inventors: Anton PERNTHANER, Palmerston North (NZ); Suzanne Hurst, Palmerston North (NZ)

(73) Assignee: KORU BIOTECH SOLUTIONS LIMITED, Palmerston North (NZ)

(21) Appl. No.: 19/482,273

(22) PCT Filed: May 10, 2024
(86) PCT No.: PCT/NZ2024/050051

§ 371 Date: Nov. 7, 2025

(30) Foreign Application Priority Data
May 10, 2023 (AU) 2023901412

(51) Int. Cl. C07K 16/1232; A61K 31/165; A61K 31/407; A61K 31/43; A61K 31/4709; A61K 31/496; A61K 31/546; A61K 31/65; A61K 31/665; A61K 31/7036; A61K 38/12; A61K 38/16; A61K 38/40; A61K 39/40; A61P 31/04; C07K 16/1203; C07K 16/1214; C07K 16/1228; C07K 16/1235

(52) U.S. Cl. CPC C07K 16/1232; A61K 31/165; A61K 31/407; A61K 31/43; A61K 31/4709; A61K 31/496; A61K 31/546; A61K 31/65; A61K 31/665; A61K 31/7036; A61K 38/12; A61K 38/168; A61K 38/40; A61K 39/40; A61P 31/04; C07K 16/1214; C07K 16/1228; C07K 16/1235; C07K 16/1242; C07K 2317/12

(57) ABSTRACT

The invention relates to methods of treating or preventing a disease or condition caused by or associated with a gram-negative bacterial infection in a subject in need thereof and compositions for use in such methods. More particularly, the present invention relates to agents and compositions, including concanavalin A, lactoferrin, colostrum and Ig from colostrum for use in the treatment or prevention of a disease or condition caused by or associated with a gram-negative bacterial infection and methods of using such agents and compositions.

### TECHNICAL FIELD

The invention relates to methods of treating or preventing a disease or condition caused by or associated with a bacterial infection in a subject in need thereof and compositions for use in such methods. More particularly, the present invention relates to agents and compositions for use in the treatment or prevention of a disease or condition caused by or associated with a gram-negative bacterial infection and methods of using such agents and compositions.

### BACKGROUND OF THE INVENTION

The following includes information that may be useful in understanding the present inventions. It is not an admission that any of the information provided herein is prior art, or relevant, to the presently described or claimed inventions, or that any publication or document that is specifically or implicitly referenced is prior art. Any discussion of the prior art throughout the specification should in no way be considered as an admission that such prior art is widely known or forms part of the common general knowledge in the field or in any particular jurisdiction.

It is well recognised that pathogenic microorganisms are causative of and/or associated with a wide variety of diseases and conditions and have a very substantial impact on both health and wellbeing, and on economic activities.

Gram-negative bacteria are causative of or associated with a number of important infections, each of which is frequently exacerbated by the release of endotoxins from the bacterial membrane.

Pneumonia, peritonitis, urinary tract infections, sepsis and bloodstream infections, persistent infections of wounds or surgical sites, and meningitis, are just some of the serious infections caused by gram-negative bacteria, such as but not limited to Escherichia coli, Serratia marcescens, Enterobacter cloacae, Salmonella spp., Pseudomonas aeruginosa, Proteus vulgaris and Klebsiella pneumoniae.

The availability of rapid and effective treatment of infections of bacterial pathogens is of particular importance with the increasing prevalence of multidrug resistance in bacterial pathogens of humans, and of other animal species. Multidrug resistant gram-negative bacteria, such as various Enterobacteriaceae, are particularly problematic, as there is a paucity of effective treatment agents for these organisms.

Another example of an important condition primarily caused by bacterial infection is mastitis, an inflammation of the mammary gland typically associated with intramammary infection. While mastitis is problematic in a number of mammalian species, including humans, the diagnosis and treatment of mastitis is of significant importance to the dairy industry. Indeed, mastitis is the costliest disease in dairy cattle. In large part, this cost is associated with the need to discard milk from cows undergoing treatment for mastitis for which a withholding period in which milk cannot be collected for consumption pertains, together with reduced milk production, including amongst cows with asymptomatic or subclinical mastitis. The cost of veterinary care of infected cows, and labour costs, are significant.

Animal wellbeing is also a serious consideration. Important gram-negative pathogens associated with mastitis in cows include coliform bacteria such as E. coli, Enterobacter spp., Klebsiella spp., and Citrobacter.

Acute bovine mastitis, like a number of diseases or conditions associated with gram-negative bacterial infections, is caused by infection with the bacteria, but is in fact initiated by lipopolysaccharide (LPS) shed from the bacterial cell wall, and the resulting inflammatory cascade and LPS-mediated enterotoxicity. Accordingly, current treatments for mastitis and other gram-negative bacterial infections must be cognizant of this risk of LPS-mediated enterotoxicity associated with such infections, which can be initiated or exacerbated by bacteriocidal treatments such as some antibiotics. Here, there is a risk that any bacteria killed by the bacteriocidal agent will release still more LPS and thereby exacerbating endotoxicity.

Accordingly, the availability of robust and effective treatments for infections of pathogenic microorganisms, including those which avoid endotoxicity or go at least some way to avoiding exacerbating endotoxicity, is of critical importance. There is a need to develop new and improved methods for treating or preventing gram-negative bacterial infections and/or the diseases or conditions they cause or with which they are associated.

It is therefore an object of the invention to provide one or more therapeutically useful agents and/or compositions useful in the treatment or prevention of a gram-negative bacterial infection and/or a disease or condition caused by or associated with a gram-negative bacterial infection, and/or to provide one or more methods of treating or preventing a gram-negative bacterial infection and/or a disease or condition caused by or associated with a gram-negative bacterial infection, or to at least provide the public with a useful choice.

### SUMMARY OF THE INVENTION

In a first aspect, the invention relates to a method of treating or preventing a disease or condition caused by or associated with a bacterial infection in a subject in need thereof, the method comprising administering to the subject an effective amount of immunoglobulin, said immunoglobulin capable of binding specifically to one or more bacterial lipopolysaccharides, wherein the immunoglobulin comprises, consists essentially of, or consists of immunoglobulin in or from colostrum.

In another aspect, the invention relates to a method of treating or preventing a disease or condition caused by or associated with a bacterial infection in a subject in need thereof, the method comprising administering to the subject an effective amount of immunoglobulin, said immunoglobulin capable of binding specifically to one or more bacterial lipopolysaccharides, wherein the immunoglobulin comprises, consists essentially of, or consists of immunoglobulin in or from colostrum.

In a further aspect, the invention relates to a method of treating or preventing a bacterial infection in a subject in need thereof, the method comprising administering to the subject an effective amount of immunoglobulin, said immunoglobulin capable of binding specifically to one or more bacterial lipopolysaccharides, wherein the immunoglobulin comprises, consists essentially of, or consists of immunoglobulin in or from colostrum.

In a further aspect, the invention relates to a method of treating or preventing a bacterial infection in a subject in need thereof, the method comprising administering to the subject an effective amount a multimerizing reagent capable of binding to one or more lipopolysaccharides from gram-negative bacteria, wherein when bound to and/or complexed with said one or more lipopolysaccharides present in the subject said multimerizing reagent is capable of further multimerization.

Any of the examples described herein can relate to any of the aspects presented herein.

In one example, the bacterial infection is an infection of gram-negative bacteria.

In a one example, the treatment or prevention comprises treatment, prevention, or amelioration of one or more symptoms of or associated with bacterial infection.

In various examples, a method contemplated herein is directed to the treatment or prevention of endotoxicity in a subject in need thereof. For example, a method as contemplated in any of the aspects discussed herein is directed to the treatment, prevention, inhibition or reversal of LPS-mediated endotoxicity in a subject. In another example, a method as contemplated in any of the aspects discussed herein is directed to the treatment, prevention, inhibition or reversal of an immunological response to LPS in a subject,

In another aspect, the invention relates to a method of treating or preventing mastitis in a subject in need thereof, the method comprising administering to the subject an effective amount of immunoglobulin, said immunoglobulin capable of binding specifically to one or more lipopolysaccharides from gram-negative bacteria, wherein the immunoglobulin comprises, consists essentially of, or consists of immunoglobulin in or from colostrum.

In another aspect, the invention relates to a method of treating or preventing mastitis in a subject in need thereof, the method comprising administering to the subject an effective amount of a multimerizing reagent capable of binding to one or more lipopolysaccharides from gram-negative bacteria, wherein when bound to and/or complexed with said one or more lipopolysaccharides present in the subject said multimerizing reagent is capable of further multimerization.

In one example, the treatment or prevention comprises treatment, prevention, or amelioration of one or more symptoms of or associated with mastitis.

In one example, the gram-negative bacteria is a bacteria selected from the group consisting of Escherichia spp., Pasteurella spp., Serratia spp., and Klebsiella spp.

In one example the effective amount is an amount effective to bind to and/or reduce endotoxicity associated with said lipopolysaccharide.

In one example, the effective amount is an amount effective to form an insoluble complex comprising lipopolysaccharide.

In one example, the immunoglobulin comprises, consists essentially of, or consists of immunoglobulin from colostrum.

In one example, the immunoglobulin comprises, consists essentially of, or consists of IgG and IgA from colostrum.

In one example, the colostrum is bovine colostrum, including pooled bovine colostrum.

In one example, the multimerizing agent is selected from the group consisting of a lectin, a protein capable of binding lipopolysaccharide, and a lipopolysaccharide-binding compound.

In one example, the multimerizing agent is a lipopolysaccharide binding protein.

In one example, the multimerizing agent is Concanavalin A.

In one example, at least some of the Concanavalin A is dimeric, for example is dimeric when administered.

In one example, prior to administration at least some of the Concanavalin A is maintained at or below pH 5.4.

In various examples, the multimerizing agent is administered together with one or more agents selected from the group consisting of: a glycoprotein, a pharmaceutically acceptable non-ionic detergent, a pharmaceutically acceptable molecular crowding agent, and a pharmaceutically acceptable capture reagent.

In one example, the multimerizing agent is administered prior to administration of one or more agents selected from the group consisting of: a glycoprotein, a pharmaceutically acceptable non-ionic detergent, a pharmaceutically acceptable molecular crowding agent, and a pharmaceutically acceptable capture reagent.

In one example, the multimerizing agent is administered after administration of one or more agents selected from the group consisting of: a glycoprotein, a pharmaceutically acceptable non-ionic detergent, a pharmaceutically acceptable molecular crowding agent, and a pharmaceutically acceptable capture reagent.

In one example, the glycoprotein is lactoferrin, for example bovine lactoferrin.

In one example, the method comprises administering to the subject an effective amount of Concanavalin A and lactoferrin.

In one example, the method comprises administering to the subject an effective amount of Concanavalin A prior to administration of an effective amount of lactoferrin.

In one example, the method comprises administering to the subject an effective amount of Concanavalin A after administration of an effective amount of lactoferrin.

In various examples, the at least one capture reagent is selected from the group consisting of: a molecular crowding agent, a polyethylene, a polyethylene glycol, and Polymyxin B.

In one example, administration to the subject comprises parenteral administration.

In one example, administration to the subject comprises topical or oral administration, or administration to a mucosal tissue.

In one example, administration to the subject comprises instillation, for example intra-mammary instillation.

In one example, administration to the subject comprises administration ex vivo.

In one example, ex vivo administration comprises administration to a sample from the subject, followed by administration of at least some of the sample to the subject.

In a further aspect the invention relates to a pharmaceutical composition comprising immunoglobulin capable of binding specifically to one or more bacterial lipopolysaccharides, wherein the immunoglobulin comprises, consists essentially of, or consists of immunoglobulin from colostrum.

In one example, the immunoglobulin comprises, consists essentially of, or consists of IgG and IgA from colostrum.

In one example, the colostrum is bovine colostrum, including pooled bovine colostrum.

In a further aspect the invention relates to a pharmaceutical composition comprising an effective amount a multimerizing reagent capable of binding to one or more lipopolysaccharides from gram-negative bacteria, wherein when bound to and/or complexed with said one or more lipopolysaccharides present in the subject said multimerizing reagent is capable of further multimerization.

In one example, the multimerizing agent is Concanavalin A.

In one example, the pharmaceutical composition comprises lactoferrin.

In one example, the lactoferrin is bovine lactoferrin.

In various examples, the pharmaceutical composition as described herein comprises one or more additional agents selected from the group consisting of: a pharmaceutically acceptable non-ionic detergent, a pharmaceutically acceptable molecular crowding agent, and a pharmaceutically acceptable capture reagent.

In one example, the capture reagent is selected from the group consisting of a lectin, a protein capable of binding lipopolysaccharide, and a lipopolysaccharide binding compound.

In one example, the pharmaceutical composition described herein for use in the treatment or prevention of a bacterial infection of gram-negative bacteria or of a disease or condition caused by or associated with a bacterial infection of gram-negative bacteria.

In a further aspect the invention relates to a pharmaceutical composition for reducing the amount of one or more lipopolysaccharides present in a subject or a sample from a subject, wherein the pharmaceutical composition comprises immunoglobulin capable of binding specifically to one or more bacterial lipopolysaccharides, wherein the immunoglobulin comprises, consists essentially of, or consists of immunoglobulin from colostrum.

In a further aspect the invention relates to a pharmaceutical composition for reducing the amount of one or more lipopolysaccharides present in a subject or a sample from a subject, wherein the pharmaceutical composition comprises a multimerizing reagent capable of binding to one or more lipopolysaccharides from gram-negative bacteria, wherein when bound to and/or complexed with said one or more lipopolysaccharides present in the subject said multimerizing reagent is capable of further multimerization.

In a further aspect the invention relates to use of immunoglobulin capable of binding to one or more lipopolysaccharides from gram-negative bacteria in the preparation of a medicament for use in treating or preventing a bacterial infection of gram-negative bacteria or of a disease or condition caused by or associated with a bacterial infection of gram-negative bacteria, wherein the immunoglobulin comprises, consists essentially of, or consists of immunoglobulin from colostrum.

In a further aspect the invention relates to use of a multimerizing reagent capable of binding to one or more lipopolysaccharides from gram-negative bacteria in the preparation of a medicament for use in treating or preventing a bacterial infection of gram-negative bacteria or of a disease or condition caused by or associated with a bacterial infection of gram-negative bacteria, wherein when bound to and/or complexed with said one or more lipopolysaccharides present in the subject said multimerizing reagent is capable of further multimerization.

In a further aspect the invention relates to a method of treating or preventing a disease or condition caused by or associated with a bacterial infection in a subject in need thereof, the method comprising administering to the subject an effective amount of an antibody-binding agent, wherein the antibody-binding reagent is capable of specifically binding one or more endogenous lipopolysaccharide-binding antibodies present in the subject.

In various examples, the antibody-binding agent is an antibody or antibody fragment, an antibody binding protein or fragment thereof, or a combination thereof.

In various examples, the antibody-binding agent is selected from the group consisting of: an anti-Ig antibody or a fragment thereof, Protein A or an antibody-binding fragment thereof, Protein G or an antibody-binding fragment thereof, an anti IgA antibody or an IgA antibody-binding fragment thereof, or an IgA-binding reagent.

In one example, the antibody-binding agent is Concanavalin A.

In a further aspect the invention relates to a method of treating or preventing a disease or condition caused by or associated with a bacterial infection in a subject in need thereof, or treating or preventing a bacterial infection in a subject in need thereof, the method comprising administering to the subject an effective amount of Concanavalin A or an analogue, derivative, or synthetic form thereof.

In one example, the method as described herein comprises administering to the subject an additional therapeutic agent.

In various examples, the additional therapeutic agent is an antibiotic selected from the group consisting of aminoglycosides, such as gentamicin, amikacin; beta-lactams; carbapenems, including imipenem, meropenem; cephalosporins, including cefotaxime, ceftazidime; chloramphenicols; fluorquinolones, such as ciprofloxacin, delafloxacin; fosfomycin; penicillins; polymyxins, such as colistin, polymyxin B; glycylcycline, such as tigecycline; sulphonamides, such as co-trimoxazole; tetracyclines, including doxycycline, eravacycline, minocycline, omadacycline; and ureidopenicillins, such as piperacillin; and any combination of two or more thereof, including combinations such as ceftolozane/tazobactam, ceftazidime/avibactam, meropenem/vaborbactam, and imipenem/cilastatin/relebactam.

In various examples, the disease or condition is selected from the group consisting of Brucellosis; Campylobacter infections; Cholera; Escherichia coli (E. coli) infections; Haemophilus influenzae infections; Klebsiella infections; Proteus infections, Legionellosis, including Legionnaires' disease; Pertussis; Plague; Pseudomonas infections; Salmonella infections; sepsis, septic shock, Shigellosis; Tularemia; Porphyromonas gingivalis infections, Heligobacter infections and Typhoid fever.

In one example, the complex comprising lipopolysaccharide comprises one or more proteins capable of binding lipopolysaccharide, such as one or more antibodies. In one example, the biological sample comprises LPS wherein at least a portion of the LPS present in the sample is bound by one or more antibodies. For example, at least a portion of the LPS is present in an LPS/antibody complex.

In various examples, the protein capable of binding lipopolysaccharide is selected from the group consisting of Protein G, Protein A, a Protein A/G conjugate, a Protein G fusion protein, a Protein A fusion protein, a Protein A/G fusion protein, an immunoglobulin-binding fragment thereof, or any combination of two of more thereof.

In various examples, the complex comprising lipopolysaccharide comprises or is bound using one or more secondary antibodies, such as one or more species-specific secondary antibodies.

In various examples, the complex comprising lipopolysaccharide and multimeric multimerizing reagent comprises or is bound using one or more secondary antibodies, such as one or more species-specific secondary antibodies.

In other examples, the complex comprising lipopolysaccharide comprises or is bound using a protein capable of binding lipopolysaccharide other than an antibody. Representative examples of such proteins capable of binding lipopolysaccharide include, for example, Protein G, Protein A, a Protein A/G conjugate, a Protein G fusion protein, a Protein A fusion protein, a Protein A/G fusion protein, an immunoglobulin-binding fragment thereof, or any combination of two of more thereof. In one example, the complex comprising lipopolysaccharide comprises or is bound using Protein G, or a Protein A/G fusion protein.

In other examples, the complex comprising lipopolysaccharide and multimeric multimerizing reagent additionally comprises or is bound using a protein capable of binding lipopolysaccharide other than an antibody. Representative examples of such protein capable of binding lipopolysaccharides include, for example, Protein G, Protein A, a Protein A/G conjugate, a Protein G fusion protein, a Protein A fusion protein, a Protein A/G fusion protein, an immunoglobulin-binding fragment thereof, or any combination of two of more thereof. In one example, the complex comprising lipopolysaccharide and multimeric multimerizing reagent additionally comprises or is bound using Protein G, or a Protein A/G fusion protein.

In one example, the subject is human.

In one example, the subject is bovine, caprine, or ovine.

In various examples, at least one of the at least one capture reagent is selected from the group consisting of a lectin, a protein capable of binding lipopolysaccharide, and a lipopolysaccharide binding compound.

In one example, the antibiotic is selected from the group consisting of cyclic peptide-comprising antibiotics.

In one example, the at least one capture reagent comprises Polymyxin B.

In one example, the at least one capture reagent comprises a molecular crowding agent.

In one example, the at least one capture reagent comprises a polyethylene or a polyethylene glycol.

In one example, the polyethylene glycol is PEG20.

In one example, the at least one capture reagent comprises Polymyxin B and one or more agents selected from the group consisting of a polyethylene or a polyethlene glycol. In one specifically contemplated example, the capture reagent comprises Polymyxin B and PEG20.

In various examples, the multimerizing agent is selected from the group consisting of a lectin, a protein capable of binding lipopolysaccharide, and a lipopolysaccharide binding compound.

In one example, the multimerizing agent is a protein capable of binding lipopolysaccharide.

In one example, the multimerizing agent is Concanavalin A.

In one example, the multimerizing agent is an albumin, such as bovine serum albumin.

In various examples, the binding reagent comprises an antibody or fragment thereof capable of selectively binding one or more components of the complex comprising lipopolysaccharide. In one example, the binding reagent comprises colostrum, milk, serum, or any combination thereof, comprising one or more antibodies.

In various examples, the composition comprises or the contacting the lipopolysaccharide with the capture reagent is performed in the presence of one or more pharmaceutically acceptable detergents.

For example, the composition comprises one or more capture reagents and a buffer comprising one or more pharmaceutically acceptable detergents, such as one or more non-ionic detergents, for example a polysorbate such as a polyoxyethylene sorbitol (e.g. polysorbate 20, polysorbate 40) or a polyoxyethylene (20) sorbitan monoalkylated.

In one example, the multimerizing reagent comprises albumin, such as bovine serum albumin.

In one example, the multimerizing reagent comprises Concanavalin A, for example the multimerizing reagent comprises Concanavalin A and wherein when administered at least some of the Concanavalin A is dimeric.

In one example, prior to administration at least some of the Concanavalin A is maintained at or below pH 5.4.

In one example, administration of Concanavalin A to the subject provides a raising of the pH, for example, to physiological pH.

In one example, at least a portion of the LPS is bound by one or more antibodies, for example, at least a portion of the LPS is present in an LPS/antibody complex.

In various examples, the antibody-binding reagent is capable of binding one or more endogenous lipopolysaccharide-binding antibodies present.

In various examples, the method comprises administering a binding reagent comprising an antibody or fragment thereof capable of selectively binding the complex comprising lipopolysaccharide analyte and multimeric Concanavalin A.

In one example, the binding reagent is or comprises polyclonal antibodies.

In various examples, the antibody-binding reagent is an antibody binding protein.

In one example, the antibody-binding reagent is selected from the group consisting of: an anti-Ig antibody or a fragment thereof, Protein A or an antibody-binding fragment thereof, Protein G or an antibody-binding fragment thereof, an anti IgA antibody or an IgA antibody-binding fragment thereof, or an IgA-binding reagent.

Other objects, aspects, features and advantages of the present invention will become apparent from the description that follows. It should be understood, however, that the detailed description and the specific examples, while indicating preferred examples of the invention, are given by way of illustration only, since various changes and modifications within the spirit and scope of the invention will become apparent to those skilled in the art from this detailed description.

### BRIEF DESCRIPTION OF THE FIGURES

The invention is exemplified in the following non limiting examples and with reference to the accompanying figures, in which:

FIG. 1 presents a graph showing that colostrum-mediated immunoglobulin protein precipitation (protein mg/mL) increases in the presence of LPS in a dose-dependent manner.

FIG. 2 presents four graphs showing that an increase in the amount of LPS/IgG complexes (FIG. 2A, FIG. 2C) and LPS/IgA complexes (FIG. 2B, FIG. 2D) detected in the precipitate complements the decrease in LPS present supernatant over a wide range of immunoglobulin concentrations and in different gram-negative pathogenic bacteria.

FIG. 3 presents two graphs presenting data from functional LAL assays, showing that immunoprecipitation by immunoglobulins removes LPS endotoxicity from the supernatant (3A) and mask LPS endotoxicity in the precipitate (3B).

FIG. 4 presents a graph showing that colostrum IgG-mediated protein precipitation (protein mg/mL) increases in a dose-dependent manner in the presence of LPS, and that this is markedly reduced when samples are boiled.

FIG. 5 presents a graph showing that precipitated LPS/IgG complexes partially retain the ability to bind to PMB.

FIG. 6 presents a graph showing that colostrum IgG reduces the endotoxicity (EU/ml) of LPS by forming LPS/IgG complexes, which is reduced after protein denaturation (boiled vs not boiled).

FIG. 7 presents two graphs showing that IgG isolated from acute mastitis milk samples is significantly better than colostrum IgG in increasing the protein concentration (7A, P<0.05) and the LPS content (7B, p<0.05) present in the precipitate.

FIG. 8 presents a graph showing that IgG isolated from colostrum is significantly better (P<0.001) in removing the endotoxic activity from the supernatant compared to IgG isolated from acute mastitis milk samples.

FIG. 9 presents a graph showing ConA precipitation by bovine lactoferrin at different concentrations of lactoferrin.

FIG. 10 presents two graphs showing LPS precipitation is mediated by ConA pH-dependent dimer to tetramer transformation for E. coli LPS (10A) and S. marcescens LPS (10B).

FIG. 11 presents two graphs showing that addition of LF improves Con A mediated precipitation of E. coli LPS (11A) and S. marcescens LPS (11B).

FIG. 12 presents a graph showing that ConA+LF treatment significantly reduces the endotoxin activity (EU/ml) from coliform acute mastitis milk samples from a diagnostic laboratory source and a farm herd source.

FIG. 13 presents a graph showing that ConA+LF significantly increases the protein concentration in precipitates from E. coli acute mastitis milk samples from a diagnostic laboratory source and a farm herd source.

FIG. 14 presents photomicrographs showing that ConA+LF mediates aggregation (clumping) of the gram-negative mastitis pathogens S. marcescens (middle row) and E. coli (bottom row), but does not cause aggregation of the gram-positive S. aureus (top row).

FIG. 15 presents two graphs showing pH buffer change from 5.6 to 7.4 enhances binding to and detection of S. marcescens LPS by concanavalin A.

FIG. 16 presents a graph showing Biotinylated Con A binding to LPS derived from various gram-negative bacteria.

FIG. 17 presents four graphs showing enhanced binding to and detection of LPS from various gram-negative bacteria by pH-mediated transformation of concanavalin A multimers.

FIG. 18 presents a graph showing precipitation of bovine lactoferrin (18A) and an SDS-PAGE image showing reduction in lactoferrin after Con A precipitation (18B).

FIG. 19 presents two graphs showing inhibition of LPS detection (19A) and inhibition of LPS-induced pro-oxidants in milk granulocytes (19B).

FIG. 20 presents three graphs showing pH-driven Con A transformation promotes E. coli LPS aggregation and the reduction of endotoxin activity. Precipitant protein content (20A), supernatant endotoxin activity (20B), and supernatant LPS levels (20C).

FIG. 21 presents photomicrographs showing that preincubation of bovine lactoferrin with gram-negative bacteria enhances their aggregation during concanavalin A multimeric transformation. Aggregation is exemplified on four mastitis pathogens. K. oxytoca (21A), E. coli (21B), S. marcescens (21C), and P. aeruginosa (21D).

FIG. 22 presents photomicrographs showing bovine colostrum Ig extracts containing IgG and IgA agglutinate E. coli (SVS 5077).

FIG. 23 presents photomicrographs showing bovine colostrum Ig extract containing IgG and IgA agglutinates gram-negative bacteria found in clinical bovine mastitis.

FIG. 24 presents two graphs showing reduction of gram-negative bacteria LPS detection after agglutination with bovine colostrum Ig (IgG/IgA) extract. Detection of LPS using anti-IgG (24A) and detection of LPS using anti-IgA (24B).

FIG. 25 presents two graphs showing the effect of milk Ig extracts on E. coli shed LPS-induced pro-oxidants in milk granulocytes. E. coli shed LPS induced pro-oxidants (25A) and effect of milk Ig extracts (25B).

### DETAILED DESCRIPTION

The present invention relates to therapeutic agents, compositions, and methods for treating or preventing a gram-negative bacterial infection and/or of treating or preventing a disease or condition associated with the presence of gram-negative bacteria in a subject.

The presence in a subject of LPS from gram-negative bacteria has long been recognised as being responsible for endotoxicity associated with gram-negative bacterial infection, and as such an important contributor to symptoms of and indeed the severity of many diseases or conditions associated with such infections.

In certain particularly contemplated example, the agents and/or compositions disclosed herein target, and the methods disclosed herein are at least partly reliant on the targeting of, lipopolysacharride (LPS) in and/or released from the cell wall of gram-negative bacteria, for example during an infection.

Importantly, the agents, compositions, and methods enable the treatment of subjects having gram-negative bacterial infections, including those at risk of developing a disease associated with such gram-negative bacterial infections, while minimising the risk of LPS-mediated endotoxicity.

Indeed, in certain circumstances, by binding to and masking the endotoxicity associated with LPS, the agents, compositions, and methods disclosed herein enable the use of existing therapies that are (in the absence of the present agents, compositions and/or methods) currently contraindicated, such as bacteriocidal agents that risk exacerbating LPS-mediated endotoxicity.

The invention thus in certain aspects relates to the applicant's development of therapeutic agents and compostions capable of binding to LPS and reducing LPS-mediated endotoxicity.

### Selected Definitions

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7). These are only examples of what is specifically intended and all possible combinations of numerical values between the lowest value and the highest value enumerated are to be considered to be expressly stated in this application in a similar manner.

Those skilled in the art will appreciate the meaning of various terms of degree used herein. For example, as used herein in the context of referring to an amount (e.g., “about 9%”), the term “about” represents an amount close to and including the stated amount that still performs a desired function or achieves a desired result, e.g. “about 9%” can include 9% and amounts close to 9% that still perform a desired function or achieve a desired result. For example, the term “about” can refer to an amount that is within less than 10% of, within less than 5% of, within less than 1% of, within less than 0.1% of, or within less than 0.01% of the stated amount. It is also intended that where the term “about” is used, for example with reference to a figure, concentration, amount, integer or value, the exact figure, concentration, amount, integer or value is also specifically contemplated.

The term “administering” as used herein refers to providing a therapeutically effective amount of an agent or composition to a subject using one or more methods of administering therapeutic agents as are known in the art. These methods comprise administering agents using oral, sublingual, intravenous, subcutaneous, transcutaneous, intramuscular, intracutaneous, intrathecal, epidural, intraocular, intracranial, inhalation, rectal, vaginal, and the like administration. In specifically contemplated examples such as those directed to the treatment or prevention of mastitis, administration by intramammary instillation is contemplated.

The term “and/or” can mean “and” or “or”.

The terms “antibody” and “immunoglobulin” or “Ig” are generally used interchangeably herein, and refer to a glycoprotein produced by the immune system in response to the presence of an antigen.

The term “antigen” means a molecule having distinct surface features or epitopes capable of stimulating a specific immune response. Antibodies (immunoglobulins) are produced by the immune system in response to exposure to antigens. Antigens maybe proteins, carbohydrates or lipids, although only protein antigens are usually classified as immunogens because carbohydrates and lipids cannot elicit an immune response on their own.

The terms “comprise”, “comprises”, and “comprising” as used in this specification and claims are not to be interpreted in an exclusive or exhaustive sense, and mean “consisting at least in part of”.

When interpreting each statement in this specification that includes the term “comprise”, “comprises”, or “comprising”, features other than that or those prefaced by the term may also be present. Related terms such as “including”, “include” and “includes” are to be interpreted in the same manner.

The term “consisting essentially of” when used in this specification refers to the features stated and allows for the presence of other features that do not materially alter the basic characteristics of the features specified.

The term “consisting of” as used herein means the specified materials or steps of the claimed invention, excluding any element, step, or ingredient not specified in the claim.

The term “Immunoglobulin A” or “IgA” means a subclass of Ig that has an important role in the immune function of mucosal surfaces, including in the context of particularly contemplated examples herein, the udder of bovine cows.

The term “Immunoglobulin G” or “IgG” means a subclass of Ig that is mostly found in circulating bodily fluids such as blood and lymph.

The term “Immunoglobulin M” or “IgM” means a subclass of Ig that is produced mainly in the spleen and is usually the first antibody to appear in response to initial exposure to an antigen.

The term “inhibit” and grammatical equivalents as used herein such as “inhibition” and “inhibiting” as used herein is used in a similar manner to “prevent” but refers to the halting of a process, for LPS-mediated endotoxicity or an immunological response to LPS in a subject, that has already begun.

The term “prevent” and grammatical equivalents as used herein such as “prevention” and “preventing” as used herein refers to the halting of a process, for example LPS-mediated endotoxicity or an immunological response to LPS in a subject, that has not yet begun. In certain examples, such prevention is for a certain period of time—for example, for so long as the concentration of the agents and/or compositions as described herein is maintained above a certain threshold. It will be appreciated that in such examples the term “prevent” does not contemplate prevention in perpetuity.

The term “reverse” and grammatical equivalents as used herein such as “reversal” and “reversing” refers to the return of a gram-negative bacterial infection or a disease or condition associated with such infection to a former or less developed state. The terms “remission” and “regression” are to be interpreted in a similar manner.

A “subject” as used herein is an animal, usually a mammal, including a mammalian companion animal or a human. Representative companion animals include feline, equine, and canine. Representative agricultural animals include bovine, ovine, caprine, cervine, and porcine. Specifically contemplated subjects are subjects which are used commercially to produce milk, such as bovine, ovine, and caprine subjects.

The term “treat” and grammatical equivalents as used herein such as “treatment” and “treating” as used herein refers to inhibiting or arresting the development of a gram-negative bacterial infection and/or of a disease or condition associated with such infection, and/or causing the amelioration, reduction, remission or regression of a gram-negative bacterial infection or a disease or condition associated with such infection or one or more symptoms or side effects thereof. Methods of assessing treatment, including methods of assessing amelioration, inhibition, arrest, reduction, remission and/or regression of disease states are known and will be apparent to a person skilled in the art.

The treatment of a gram-negative bacterial infection or a disease or condition associated with such infection, and/or the prevention, inhibition or reversal of processes associated with a gram-negative bacterial infection or a disease or condition associated with such infection may manifest in a number of ways. For example, in some examples the treatment, prevention, inhibition or reversal of processes such as for example LPS-mediated endotoxicity or an immunological response to LPS in a subject, leads to clearance of the infection, and/or leads to slowing of disease progression, and/or improved quality of life in subjects with the disease.

In various examples the treatment, prevention, inhibition or reversal of processes associated with a gram-negative bacterial infection or a disease or condition associated with such infection manifests as decreased endotoxicity, and/or decreased infection (for example a reduction in the number of bacterial present in a subject). In other examples the treatment, prevention, inhibition or reversal of processes associated with a gram-negative bacterial infection or a disease or condition associated with such infection manifests as an increased rate of survival of a subject.

Those skilled in the art will recognise, on reading this description, that various uses of such methods in the treatment or prevention of infections of gram-negative bacteria and/or a disease or condition caused by or associated with an infection of gram-negative bacteria, and particularly pathogenic gram-negative bacteria, are provided.

Accordingly, in one aspect, the invention relates to a method of treating or preventing a disease or condition caused by or associated with a bacterial infection in a subject in need thereof, the method comprising administering to the subject an effective amount of immunoglobulin, said immunoglobulin capable of binding specifically to one or more bacterial lipopolysaccharides, wherein the immunoglobulin comprises, consists essentially of, or consists of immunoglobulin in or from colostrum.

Thus, in certain examples, the therapeutic agent is, the composition comprises, or the treatment comprises the administration of, immunoglobulin from colostrum.

### Colostrum

Colostrum is produced by most mammals just prior to giving birth. It is a nutrient- and bioactive-rich ‘first milk’, shown in various species to comprise immune cells, immunoglobulins, growth factors and cytokines. Other bioactives, including lactoferrin, lysozyme, lactoperoxidase, and anti-inflammatory bioactives have also been shown to be present in colostrum.

Without wishing to be bound by any theory, the inventors believe that, despite not being quite as effective in precipitating LPS from solution in the model systems exemplified herein in the Examples as immunoglobulins from mastitis milk samples, immunoglobulins from colostrum are superior in removing and/or masking the endotoxic activity associated with LPS. While bovine colostrum has been extensively characterized herein in the Examples, the inventors believe, again without wishing to be bound by any theory, that any source of colostrum will provide effective LPS binding and aggregation.

Particularly convenient sources of colostrum include those from dairy animals, such as caprine, ovine, camelids, equine, and bovine animals.

In another aspect, the invention relates to a method of treating or preventing a bacterial infection in a subject in need thereof, the method comprising administering to the subject an effective amount a multimerizing reagent capable of binding to one or more lipopolysaccharides from gram-negative bacteria, wherein when bound to and/or complexed with said one or more lipopolysaccharides present in the subject said multimerizing reagent is capable of further multimerization.

Thus, in certain examples, the therapeutic agent is, the composition comprises, or the treatment comprises the administration of, a multimerizing agent, such as Concanavalin A.

### Administration and Formulation

It will be apparent to a person skilled in the art that the formulation of the agents and/or compositions described herein will depend on the method of administration. For example, the agents and/or compositions described herein are in certain examples formulated as creams, lotions, tablets, capsules, pellets, dispersible powders, granules, suppositories, syrups, elixirs, lozenges, injectable solutions, sterile aqueous or non-aqueous solutions, suspension or emulsions, patches and the like.

For example in various examples the agents and/or compositions described herein are formulated as tablets, capsules, pellets, dispersible powders, granules, solutions, syrups, suspensions or emulsions.

In exemplary examples, the agents and/or compositions described herein are formulated for instillation, for example intramammary instillation.

In other examples, the agents and/or compositions described herein are formulated as a solid dosage form, such as tablets, capsules or pellets.

The formulations that are suitable for a particular method of administration will be apparent to those skilled in the art.

As will be readily appreciated by those skilled in the art, the route of administration and the nature of the pharmaceutically acceptable carrier will depend on the nature of the condition and the mammal to be treated. It is believed that the choice of a particular carrier or delivery system, and route of administration could be readily determined by a person skilled in the art. In the preparation of any formulation containing the agents and/or compositions described herein, care should be taken to ensure that the activity of the agent or composition is not destroyed in the process and that the agent or composition is able to reach its site of action without being destroyed. In some circumstances it may be necessary to protect the agent or composition by means known in the art, such as, for example, microencapsulation. Similarly, the route of administration chosen should be such that the agent or composition reaches its site of action.

Those skilled in the art may readily determine appropriate formulations for the agents and/or compositions described herein using conventional approaches. Identification of preferred pH ranges and suitable excipients, for example antioxidants, is routine in the art, and examples of preferred pH ranges for specific agents and/or compositions described herein are provided in the Examples. Buffer systems are routinely used to provide pH values of a desired range and include carboxylic acid buffers for example acetate, citrate, lactate and succinate. A variety of antioxidants are available for such formulations including phenolic compounds such as BHT or vitamin E, reducing agents such as methionine or sulphite, and metal chelators such as EDTA.

The agents and/or compositions described herein may be prepared in parenteral dosage forms, including those suitable for intramammary, intravenous, intrathecal, and intracerebral or epidural delivery. The pharmaceutical forms suitable for injectable use include sterile injectable solutions, suspensions, or dispersions, instillable solutions, suspensions, or dispersions, and sterile powders for the extemporaneous preparation of sterile injectable or instillable solutions, suspensions, or dispersions. They should be stable under the conditions of manufacture and storage and may be preserved against reduction or oxidation and the contaminating action of microorganisms such as bacteria or fungi.

The solvent or dispersion medium for the injectable solution, suspension, or dispersion may contain any of the conventional solvent or carrier systems for compound actives, and may contain, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about where necessary by the inclusion of various antibacterial and antifungal agents, for example, parabens, chlorobutanol, phenol, sorbic acid, thimerosal and the like. In many cases, it will be preferable to include agents to adjust osmolality, for example, sugars or sodium chloride. In various examples, the formulation for injection will be isotonic with blood. Prolonged absorption of the injectable or instillable compositions can be brought about by the use in the compositions of agents delaying absorption, for example, aluminium monostearate and gelatin. Pharmaceutical forms suitable for injectable or instillable use may be delivered by any appropriate route including intramammary, intravenous, intramuscular, intracerebral, intrathecal, epidural injection or infusion.

Sterile injectable solutions are prepared by incorporating the active agent in the required amount in the appropriate solvent with various of the other ingredients such as those enumerated above, as required, followed by filtered sterilization. Generally, dispersions or suspensions are prepared by incorporating the various sterilised active ingredient into a sterile vehicle which contains the basic dispersion medium and the required other ingredients from those enumerated above. In the case of sterile powders for the preparation of sterile injectable or instillable solutions, preferred methods of preparation are vacuum drying or freeze-drying of a previously sterile-filtered solution of the active ingredient plus any additional desired ingredients.

Other pharmaceutical forms include oral and enteral formulations, in which the active agent may be formulated with an inert diluent or with an assimilable edible carrier, or it may be enclosed in hard or soft shell gelatin capsule, or it may be compressed into tablets, or it may be incorporated directly with the food of the diet. For oral therapeutic administration, the active agent may be incorporated with excipients and used in the form of ingestible tablets, buccal or sublingual tablets, troches, capsules, elixirs, suspensions, syrups, wafers, and the like. The amount of active agent in such therapeutically useful compositions is such that a suitable dosage will be obtained.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active agent(s) may be incorporated into sustained-release preparations and formulations, including those that allow specific delivery of the active agent to specific regions of the gut.

Liquid formulations may also be administered enterally via a stomach or oesophageal tube.

Enteral formulations may be prepared in the form of suppositories by mixing with appropriate bases, such as emulsifying bases or water-soluble bases. It is also possible and for specific diseases or infections is specifically contemplated for the agents and/or compositions described herein to be administered intramammarily, topically, intranasally, intravaginally, intraocularly and the like.

Other forms suitable for administration, for example topical application such as creams, lotions and gels, or compositions suitable for inhalation or intranasal delivery, for example solutions, dry powders, suspensions or emulsions, are also contemplated.

The agents and/or compositions described herein may be administered by inhalation in the form of an aerosol spray from a pressurised dispenser or container, which contains a propellant such as carbon dioxide gas, dichlorodifluoromethane, nitrogen, propane or other suitable gas or combination of gases. The agents and/or compositions may also be administered using a nebuliser.

Pharmaceutically acceptable vehicles and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

In certain circumstances, for example for use in the treatment of particular subjects, it is especially advantageous to formulate the compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutically acceptable vehicle. The specification for the dosage unit forms contemplated herein are dictated by and directly dependent on (a) the characteristics of the active agent and the particular therapeutic effect to be achieved, and (b) the limitations inherent in the art of compounding active materials for the treatment of disease in living subjects having a diseased condition in which bodily health is impaired as herein disclosed in detail.

As mentioned above the principal active agent may be compounded for convenient and effective administration in therapeutically effective amounts with a suitable pharmaceutically acceptable vehicle in dosage unit form. A unit dosage form can, for example, contain the principal active agent in amounts ranging from 0.25 μg to about 2000 mg or more. Expressed in proportions, the active compound may be present in from about 0.25 μg to about 2000 mg/mL of carrier. In the case of compositions containing supplementary active ingredients, the dosages are determined by reference to the usual dose and manner of administration of the said ingredients.

The term “therapeutically effective amount” as used herein refers to a dose of an agent or composition sufficient to provide a concentration high enough to effect the desired result. For example, in certain examples a therapeutically effect amount is a dose of an agent described herein sufficient to result in one or more of the following; the treatment, prevention, inhibition or reversal of a gram-negative bacterial infection and/or of a disease or condition caused by or associated with such infection, including one or more symptoms thereof or process associated therewith, such as LPS-mediated endotoxicity.

The therapeutically effective amount of an agent will in certain examples be affected by a number of factors and can be adjusted based on these factors. For example, the therapeutically effective dose may be affected by the bodyweight of the subject, metabolic capacity and synergy between combinations of actives administered. The dose that can be administered to a subject may also be affected by other factors such as interactions with other medicines that the subject is taking and severity of/ability to tolerate any side effects of the agents and/or compositions administered.

In some examples, the desired result to be achieved by the therapeutically effective amount comprises the slowing of disease progression, an improvement in the quality of life of the subject and/or an increased rate of survival.

In various examples, the desired result to be achieved by the therapeutically effective amount is the amelioration of one or more symptoms associated with a gram-negative bacterial infection and/or of a disease or condition associated with such infection, such as sepsis, organ failure, and the like.

Also contemplated herein is a pharmaceutical composition comprising a therapeutically effective amount of an agent as hereinbefore defined, or a pharmaceutically acceptable salt thereof, together with at least one pharmaceutically acceptable carrier or diluent.

The term “composition” is intended to include the formulation of an active ingredient with a carrier, such as an encapsulating material as carrier, to give a capsule in which the active ingredient (with or without other carrier) is surrounded by carriers.

The agents described herein are in certain examples administered individually, and in other examples are administered in combination, either with other agents described herein, or with other therapeutic agents or both. The combination may allow for separate, sequential or simultaneous administration of the compound as hereinbefore described with the other active ingredient(s). The combination may be provided in the form of a pharmaceutical composition.

For example, in some examples two or more agents described herein are administered to a subject together. When administering combinations of agents to a subject, the dose of each individual agent may be less than the therapeutically effective amount such that the combined dose of the two or more agents is equal to or greater than the therapeutically effective amount.

Furthermore, in some examples one or more agents described herein is administered to a subject together with one or more additional agents. The additional agents may be, for example, agents that treat, inhibit and/or reverse a gram-negative bacterial infection and/or of a disease or condition associated with such infection, and/or causing the amelioration, reduction, remission or regression of a gram-negative bacterial infection or a disease or condition associated with such infection or one or more symptoms or side effects thereof, or ameliorate, reduce or reverse one or more processes associated with such infections or disease, such as the immunological response to gram-negative bacterial infection.

In certain examples, the agents and/or compositions contemplated herein are administered together with one or more immunomodulators.

In certain examples, the agents, compositions, and methods described herein enable the use of therapies and/or therapeutic regimens that may otherwise be contraindicated, for example due to an increased risk of liberating LPS and attendant endotoxicity, such as that that may occur when bacteriocidal agents such as some antibiotics are used. In such examples, the agents, compostions and methods disclosed herein can be utilised in combination with, for example, the administration of one or more antibiotics effective to treat gram-negative bacterial infections. For example, particularly contemplated therapeutic methods comprises administration of one or more agents selected from the group consisting of aminoglycosides, such as gentamicin, amikacin; beta-lactams; carbapenems, including imipenem, meropenem; cephalosporins, including cefotaxime, ceftazidime; chloramphenicols; fluorquinolones, such as ciprofloxacin, delafloxacin; fosfomycin; penicillins; polymyxins, such as colistin, polymyxin B; glycylcycline, such as tigecycline; sulphonamides, such as co-trimoxazole; tetracyclines, including doxycycline, eravacycline, minocycline, omadacycline; and ureidopenicillins, such as piperacillin; and any combination of two or more thereof, including combinations such as ceftolozane/tazobactam, ceftazidime/avibactam, meropenem/vaborbactam, and imipenem/cilastatin/relebactam.

In some examples the one or more additional agents may for example be agents that reduce side effects associated with the agents described herein.

When one or more agents described herein are administered in combination with one or more additional agents, the dose of each agent administered may be less than the dose that would be administered if the agents were administered separately.

In some examples when one or more agents described herein are administered in combination with one or more additional agents, the dose of each agent administered may be greater than the dose that would be administered if the agents were administered separately. For example, this may be the case if the one or more additional agents leads to reduced side effects, allowing a greater dose to be tolerated.

It will be appreciated that the likelihood that treatment of a bacterial infection or a disease or condition caused by or associated with a bacterial infection is successful will usually be heavily time-dependent, and particularly on the speed with which an effective amount of a therapeutic agent can be administered to a subject in need thereof. Indeed, early diagnosis or detection of disease will in many examples allow for rapid treatment, and in certain circumstances preventative treatment. In other circumstances, disease prevalence, such as the identification of the presence of disease-causing bacteria in a particular locus, environment, or population, supports prophylactic treatment.

In certain examples however, even when disease-causing bacteria has been identified as being present in a population or environment, there may be a reluctance to prophylactically treat one or more subjects not yet manifesting infection or symptoms of infection. Bovine mastitis is one such disease, where prophylactic treatment of all individuals in a herd would in many cases lead to reduced productivity and is thus generally avoided if possible.

Accordingly, in certain examples, the rapid detection of bacterial infection and/or rapid treatment with one or more agents and/or compostions as herein described is particularly contemplated. For example, application of colostrum immunoglobulins to the udder of bovine subjects following the detection of LPS-inducing acute mastitis would ensure that LPS-specific antibodies are immediately available to neutralise LPS as it becomes released from either degraded milk proteins (i.e., casein) by inflammatory proteases or shed from invading gram-negative bacteria. Without wishing to be bound by any theory, the inventors believe a rapid prophylactic approach such as this is likely to reduce the overall LPS endotoxicity and disease impact, and in turn will facilitate or enable the subject's own immune system to respond.

### Diagnosis of Disease and Monitoring Treatment

Advantageously, the diagnosis of a disease or condition in a subject or the identification of a subject at increased risk of having or developing a disease or condition, wherein the disease or condition is caused by or associated with the presence of a gram-negative bacteria, will in certain examples be useful to inform and/or monitor the therapeutic approach or regimen used to treat (including to control, reverse, mitigate and/or prevent) such a disease or condition in the subject.

Thus, in certain examples the therapeutic methods disclosed herein involve or are informed by information provided by one or more diagnostic methods, such as a method that directly or indirectly determines the presence of LPS in a sample, such as a sample obtained from a subject being treated.

For example, LPS can be detected using a variety of assays, such as the endotoxicity assays exemplified herein, or an immunological assay, such as ELISA as herein exemplified, or Western Blots, Lateral Flow tests and Biosensors. ELISA tests have the advantage of being comparatively fast and accurate and the tests are amenable to high throughput and automation. Lateral flow tests are advantageously employed where there is no expectation of having ready access to the laboratory equipment necessary for ELISA or other methods, such as in workplace or public event screening and the like. The rapid provision of results from lateral flow tests also enable rapid diagnoses, which in turn can ensure better treatment decisions and/or health outcomes, and can frequently avoid the need for time-consuming or costly sample logistics and laboratory analysis.

### Gram-Negative Bacterial and Bacterial Pathogens

Gram-negative bacteria present one of the world's most significant public health problems due to their high resistance to antibiotics. These microorganisms have significant clinical importance in hospitals because they put patients present in the intensive care unit (ICU) at high risk and lead to high morbidity and mortality. Two large groups of gram-negative bacteria, the Enterobacteriaceae and the non-fermenters, are responsible for most clinically relevant isolates. Other gram-negative organisms of clinical relevance exist, including but not limited to Neisseria, Haemophilus spp., Helicobacter pylori, and Chlamydia trachomatis. Gram-negative bacteria suitable for treatment using the methods and compositions described herein thus include the Enterobacteriaceae, Pasteurellaceae and Aeromonas groups.

### Enterobacteriaceae

Enterobacteriaceae are a heterogeneous group widely dispersed in nature. They account for about 80% of gram-negative isolates with a myriad of disease-causing species in humans and other animals, including urinary tract infections, pneumonia, diarrhea, meningitis, sepsis, endotoxic shock, and many others. Species of particular concern and which frequently affect humans include Escherichia, Proteus, Enterobacter, Klebsiella, Citrobacter, Yersinia, Shigella, and Salmonella, among others.

Characteristics of Enterobacteriaceae typically employed in laboratory characterization include their being bacilli, non-sporulated, and having variable motility, are able to grow in the presence and absence of oxygen, are able to ferment glucose, are cytochrome oxidase negative, and can reduce nitrate to nitrite.

### Non-Fermenters

The non-fermenter gram-negative bacilli are usually encountered in the clinic than Enterobacteriaceae. However, they are clinically relevant since they cause severe, fatal infections, especially in a hospital environment. They also cause opportunistic diseases in ICU patients who undergo invasive procedures. The main non-fermenter gram-negative microorganisms that cause human disease are Pseudomonas aeruginosa, Acinetobacter baumannii, Burkholderia cepacia, Burkholderia pseudomallei, Stenotrophomonas spp., Alcaligenes spp., and Moraxella spp. These are characterised by being aerobic and non-sporulated, are incapable of fermenting sugars and instead use sugars via oxidative metabolism.

The methods and related aspects are useful in the treatment of gram-negative bacterial infections and finds particular application in the treatment of gram-negative bacterial pathogens and as a result the treatment and/or prevention of diseases or conditions caused by or associated with gram-negative bacterial infections.

Gram-negative bacteria can cause serious infections and are able to reach almost all systems in the subject organism, including the digestive system, nervous system, urinary system, and bloodstream. These microorganisms readily colonize the intestines, airways, and skin, thereby favouring the spread to other parts of the organism, especially in immunocompromised individuals. Gram-negative bacteria cause infections including pneumonia, peritonitis (inflammation of the membrane that lines the abdominal cavity), urinary tract infections, bloodstream infections including sepsis, wound or surgical site infections, and meningitis.

Nosocomial infections of the lower respiratory tract are particularly challenging to treat. This is because the pathogenic gram-negative bacteria typically involved are not only responsible for a good portion of these infections, they are non-responsive to antibiotic therapy due to the high resistance rates and the poor penetration of drugs into the lung parenchyma.

Another major concern is gastroenteritis caused by Enterobacteriaceae, particularly Shigella spp., Salmonella spp., and enteropathogenic E. coli. Gastroenteritis affects millions of people worldwide and is generally related to a lack of sanitation. Bacterial meningitis—a potentially fatal disease if not treated in time—is likewise a major concern both in the community and in the hospital environment. Urinary tract infections are also common, especially in young women. However, these infections became a problem with the widespread emergence of multi-resistant bacteria. Bacteremia is an important complication of these infections because of the acquisition of resistance.

Gram-negative pathogenic bacterial infections suitable for treatment using the methods and related aspects disclosed herein include the following: Brucellosis; Campylobacter infections; Cholera; Escherichia coli (E. coli) infections; Haemophilus influenzae infections; Klebsiella infections; Mastitis; Proteus infections, Legionellosis, including Legionnaires' disease; Pertussis; Plague; Pseudomonas infections; Salmonella infections; Shigellosis; Tularemia; Porphyromonas gingivalis infections, Heligobacter infections and Typhoid fever.

Certain gram-negative bacteria of particular clinical concern are discussed below.

Brucella infections, frequently referred to as Brucellosis, are sometimes asymptomatic but most commonly result in acute illness, with symptoms including fever, arthralgia, headache, malaise, anorexia, constipation, respiratory tract symptoms and hepatosplenomegaly observed. If not adequately treated, chronic and persistent infections in joints, bone, liver or spleen can result. Brucellosis is often seen in New Zealand in those working with livestock, while internationally the ingestion of unpasteurized goat's cheese is the most common risk factor.

Campylobacter bacteria, usually Campylobacter jejuni, cause inflammation of the colon (colitis) that results in fever and diarrhoea. These bacteria are a common cause of infectious diarrhoea, and are notifiable diseases/diseases of public health interest in a number of countries.

In many countries, all strains of Vibrio cholerae (the causative pathogen of Cholera) are notifiable despite only isolates 01 and 0139 having the potential to produce cholera toxin being associated with clinical cholera. Cholera is characterised by vomiting and potentially severe diarrhoea, which may lead to profound dehydration and death. Methods to treat Vibrio cholera are thus of significant interest, particularly in countries in which this pathogen is endemic or widespread.

Many strains of E. coli are harmless and/or non-pathogenic, being part of the normal gut microflora of mammals including humans. However, pathogenic strains of E. coli are frequently the causative agent of enteric disease and associated diarrhoea or dysentery, while other pathotypes cause extra-intestinal infections such as urinary tract infections or meningitis.

Legionella bacteria are ubiquitous in many environments, particularly in soil and aquatic environments, and Legionella infections are a common cause of pneumonia. Most cases in New Zealand are caused by L. longbeachae and L. pneumophila. Legionellosis is more common in older people, smokers, chronic disease sufferers and the immunocompromised. The most common clinical manifestation of Legionellosis reported worldwide is Legionnaires' disease, but non-pneumonic disease (such as Pontiac fever, an acute febrile illness usually accompanied by cough), and extrapulmonary disease, which usually manifest as infection of the skin, joints, pericardium or other organs, are thought to be often clinically un- or mis-diagnosed, and thus may be underreported.

Sepsis and septic shock are life-threatening conditions caused by one or more unregulated host responses to infection. Pathogenic gram-negative bacteria, including E. coli, Klebsiella pneumoniae, Acinetobacter baumannii, Enterobacter cloaceae, Proteus mirabilis, and Pseudominas aeruginosa, are common causative agents of sepsis and septic shock. Sepsis most commonly occurs as a result of infections in the urinary tract, the lungs, or the peritoneum, but infections in other organs including skin, the soft tissue, and the CNS are observed. Septic shock is usually considered to be a subset of sepsis in which profound abnormalities in the circulatory system, in cellular processes or in metabolic pathways are observed and are associated with an increased risk of mortality.

In principle, any disease or condition associated with a gram-negative bacterial source of LPS is amenable to treatment using the methods and associated aspects disclosed herein, although the treatment of pathogenic bacterial infections and associated diseases such as those discussed above is a focus of the present invention.

Particularly contemplated herein are therapeutic agents, methods, and compositions comprising or utilising colostrum immunoglobulins, and/or one or more multimerizing agents such as ConA, as are capable of binding to and/or masking the endotoxic activity of gram-negative lipopolysaccharides, as described herein.

### Lipopolysaccharides

Lipopolysaccharides (LPS, often referred to as endotoxin) are carbohydrates present in the outer membrane of Gram-negative bacteria, generally comprising a 0-antigen, an inner oligosaccharide core, and an outer core. LPS has been reported to replicate septic shock when experimentally administered to mammals including humans, and has been reported to be a major factor in gram-negative sepsis. Similarly, LPS has been reported to replicate symptoms of mastitis when experimentally administered.

The O-antigen is a repetitive glycan polymer attached to the core oligosaccharide and is the outermost component of LPS molecules. The composition of the O-antigen varies from species to species and strain to strain, with over 160 different 0-antigen structures reported for E. coli strains alone. It has been reported that the O-antigen is the most variable portion of the LPS molecule, and most relevant to antigenic specificity.

The core oligosaccharide comprises sugars, commonly heptose and mannose derivatives such as 3-Deoxy-D-manno-oct-2-ulosonic acid (also known as keto-deoxyoctulosonate (KDO)), and often other non-carbohydrate components including amino acids or phosphates.

Lipid A is a hydrophobic fatty acid-containing molecule that anchors the LPS in the bacterial membrane. Typically, lipid A is a phosphorylated glucosamine disaccharide comprising multiple fatty acids. The particular composition of lipid A can differ across bacterial species and stains, though it is the most conserved component of LPS.

LPS has been reported to bind the CD14/TLR4/MD2 receptor complex in many animal cell types. From a health perspective, the CD14/TLR4/MD2 receptor-positive cells of particular interest include monocytes, dendritic cells, macrophages, and B cells, the binding of LPS to which promotes the secretion of pro-inflammatory cytokines, nitric oxide, and eicosanoids.

In certain examples, a capture reagent binds to LPS or to LPS/antibody complexes. In various examples, the at least one capture reagent is selected from the group consisting of a lectin, a protein capable of binding lipopolysaccharide, and a lipopolysaccharide binding compound.

In certain examples, the capture reagent is an antibiotic, such as a cyclic peptide-comprising antibiotic. In one particularly contemplated example, the capture reagent comprises Polymyxin B.

In one example, the capture reagent comprises a molecular crowding agent. In one example, the molecular crowding reagent is a polyethylene glycol.

The multimerizing reagent is capable of binding lipopolysaccharide or to LPS/antibody complexes present in the sample and/or captured by the at least one capture reagent. Without wishing to be bound by any theory, the inventors believe that multimerization of LPS-bound multimerizing reagent results in a configuration of LPS-containing complex that provides for enhanced binding and/or masking by binding reagents such as antibodies, whether endogeneous antibodies present in the sample, or exogeneous antibodies added to the assay.

In various examples, the multimerizing reagent is selected from the group consisting of a lectin, a protein capable of binding lipopolysaccharide, a lipopolysaccharide binding compound, molecules that binds to the lipopolysaccharide-capture reagent complex, such as albumin or ovalbumin, and an antibiotic.

In certain examples, the multimerizing agent is a protein capable of binding lipopolysaccharide, such as lipopolysaccharide binding protein, or bovine serum albumin.

In one example, the LPS and/or complexes comprising LPS is bound, for example, using as a binding reagent an immunoglobulin, such as a species-specific, anti-Ig secondary antibody. In another example, the LPS is bound using a protein capable of binding lipopolysaccharide, such as Protein G.

In one example, anti-LPS antibodies (and/or complexes comprising anti-LPS antibodies) are bound, for example, using species-specific, anti-Ig secondary antibodies. In another example, anti-LPS antibodies are bound using an immunoglobulin-binding protein, such as Protein G, Protein A, a Protein A/G conjugate, a Protein G fusion protein, a Protein A fusion protein, a Protein A/G fusion protein, an immunoglobulin-binding fragment thereof, or any combination of two of more thereof.

In a particularly contemplated example, the multimerizing agent is Concanavalin A.

### Concanavalin A

Concanavalin A (ConA) is a plant-based lectin that has specific binding properties for mannosylated glycoproteins. As shown herein in the Examples, ConA directly binds to glycoproteins found on shed LPS.

Without wishing to be bound by any theory, the inventors believe that the observed specificity of this binding may at least in part be due to the unique structural plasticity of ConA, which is absent in other related plant lectins. ConA in solution is able to change from a simple dimer structure to a complex tetramer with specific changes in pH. Importantly, this pH dependent transformation coincides with an increase in the number of binding sites for mannosylated glycoproteins. As exemplified herein, this pH-dependent structural transformation of ConA has been applied and found to enhance the antibody-based binding of both free LPS and LPS/Ig complexes in biological samples (e.g., milk, urine, plasma).

In various examples, the ConA is a ConA analogue, ConA derivative of fragment, or synthetic ConA.

### Lactoferrin

Lactoferrin (LF) is a mammalian innate host-defence protein that exhibits anti-microbial activity. In adult bovine milk, LF occurs in small quantities ranging from 0.1 to 0.3 mg/mL. Structurally, LF is a single polypeptide that forms two homologous globular domains, one of which is heavily glycosylated. Bovine LF, in particular, contains mannose residues within this glycosylated domain. Furthermore, the globular configuration of LF also creates regions of positive charge amino acids that are expected, without wishing to be bound by any theory, to be capable of associating with and possibly binding to negatively charged moieties, such as negatively-charged regions of proteins and other biological molecules, such as LPS as has been shown herein in the Examples.

The phrase “under conditions suitable for multimerization of the multimerizing reagent” as used herein include physiological conditions, for example at physiological pH. In certain examples, such conditions are provided by appropriate formulation of the composition, for example as a buffered solution, typically one comprising BSA, bovine gamma globulin (BGG) or phosphate buffered saline (PBS) with or without Tween or another ionic detergent. These added agents also tend to assist in the reduction of nonspecific binding. In certain examples, such as examples in which the multimerizing reagent is Concanavalin A, conditions suitable for multimerization of the multimerizing reagent will typically include a pH of at least about 7, such as a pH of 7.2 or greater.

The phrase “under conditions suitable for the formation of a complex comprising lipopolysaccharide analyte and multimeric multimerizing reagent” as used herein include physiological conditions, such as those provided by administration of a buffered composition, typically one comprising BSA, bovine gamma globulin (BGG) or phosphate buffered saline (PBS) with or without Tween or another ionic detergent. As above, these added agents also tend to assist in the reduction of nonspecific binding.

### Additional Agents

While examples of the methods and compositions contemplated herein employ antibodies, including species-specific antibodies, alternative agents capable of binding to LPS-containing complexes in which an antibody is present are also suitable for use. Specifically contemplated examples include proteins capable of binding lipopolysaccharides, such as Protein A, Protein G, and Protein A/G, each of which have been used extensively for the binding to and/or purification of antibodies. While full length Protein A or Protein G can be used, in the context of antibody binding a truncated recombinant form is typically used.

Protein A (SpA) in Staphylococcus aureus is encoded by the spa (staphylococcal protein A) gene. SpA is a 42 kDa protein comprising several regions with different functions: The signal sequence (S region) in the N-terminal part is followed by four or five highly homologous immunoglobulin G (IgG)-binding domains in tandem (the E, D, A, B, and C regions). The C-terminal region, also referred to as the X region, has two domains: (i) a repeat region XR, consisting of variable repeats with mostly octapeptide structures, and (ii) the XC region, consisting of a conserved sequence including an LPXTG-binding motif, which confers anchoring to the cell wall. It has been reported that SpA interacts with human IgG by binding to the Fc part of the immunoglobulin, and further that SpA can bind to other host structures, such as the von Willebrand factor to promote adhesion to platelets.

Protein G originates in group C and G Streptococcal bacteria. The native protein is a 56-kDa or 58-kDa polypeptide with multiple binding sites for immunoglobulins as well as a binding site for albumin. The most commonly used recombinant form of the protein is a truncated version that has the albumin binding site removed but that retains the IgG-binding capabilities of the native molecule. Protein G binds to antibodies through the heavy chains in the region of the Fc fragment, but at a different site than that of Protein A. Protein A and Protein G exhibit different antibody binding specificities and affinities, and thus offer options in binding and purifying antibodies, depending on the type of antibody desired.

A chimeric fusion protein consisting of the combination of Protein A and Protein G, called Protein A/G, merges the advantages of both protein specificities into one molecule. As a consequence, Protein A/G is often the protein capable of binding lipopolysaccharide of choice herein.

Since most IgG type antibodies bind to Protein A, G, or A/G via their Fc fragments on the heavy chains, the antigen binding sites at the ends of the Fab fragments remain open to interact with antigens. As a consequence, binding of antigen-bound IgG antibodies (i.e., IgG antibody:antigen complexes) is readily achieved using these proteins capable of binding lipopolysaccharides.

In certain examples, for example in methods described herein involving diagnosis of disease or monitoring disease progression or treatment efficacy, detectably-labelled Protein G, Protein A, and Protein A/G, typically being recombinant forms of each, are used. These are readily available from commercial suppliers. For example, horseradish peroxidase conjugated Protein G (HRP-Protein G, cat #M00090) as used herein in the Examples was sourced from GenScript (NJ, USA), with HRP-Protein A (cat #M00089) also being available from this supplier. ThermoFisher Scientific supplies a range of recombinant Protein A/G and Protein G conjugates suitable for the detectable binding of antibodies, including peroxidase- or alkaline phosphatase-conjugated Protein A/G and Protein G, with biotinylated conjugates also being available from the same supplier, while fluorescein conjugated Protein G is available, for example from Rockland Immunochemicals Inc., PA, USA.

The invention is further described with reference to the following examples. It will be appreciated that the invention as claimed is not intended to be limited in any way by these examples.

### EXAMPLES

### Example 1: Assessment of Bovine Colostrum as a Therapeutic Agent

This example presents experiments assessing the efficacy of bovine colostrum in reducing LPS endotoxicity associated with gram-negative bacterial infections.

### Methods and Materials

Colostrum was sourced from a commercial supplier.

Gram-negative mastitis pathogens including Escherichia coli, Serratia marcescens, Klebsiella oxytoca, Pasteurella multicida, Pseudomonas aeruginosa, Proteus vulgaris and Salmonella typhimurium were isolated from acute mastitis milk samples sourced from New Zealand.

E. coli LPS was prepared in house from a well characterised acute mastitis E. coli isolate.

LPS precipitation experiments were conducted with various concentrations of colostrum, ranging from 1:5 to 1:2360. E. coli shed LPS (ng/mL) was incubated with a 0.5 mg/mL bovine colostrum Ig extract for 1 h at 37° C. The mixture was centrifuged at 6000×g and the precipitated pellet was resuspended in Tris/HCl buffer and left at 4° C. or boiled (>98° C.) for 10 mins to denature proteins. The boiled or un-boiled precipitated pellet (i.e. LPS/Ig complexes) were assessed by ELISA for endotoxin activity using LAL bioassay, LPS/Ig complexes and release of LPS after boiling the pellet. LPS endotoxicity experiments were performed according to instructions of the manufacturer (GenScript Biotech Corporation).

### Results

Immunoglobulins prepared from the bovine colostrum pool recognised LPS derived from a representative range of gram-negative mastitis pathogens including Escherichia coli, Serratia marcescens, Klebsiella oxytoca, Pasteurella multicida, Pseudomonas aeruginosa, Proteus vulgaris and Salmonella typhimurium.

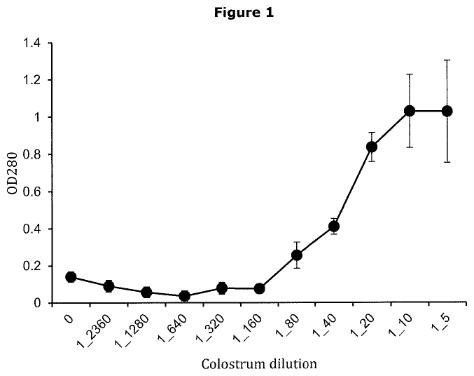

The ability of this immunoglobulin preparation to inactivate endotoxin was then assessed in vitro on E. coli LPS. Immunoprecipitation experiments were developed for a defined E. coli LPS concentration of 12.5 ng/ml with a range of immunoglobulin dilutions containing a mixture of IgG and IgA. These experiments demonstrated that LPS could interact with and precipitate immunoglobulins (and accordingly, vice versa) between a colostrum dilution of 1:80 and 1:5, as shown in FIG. 1.

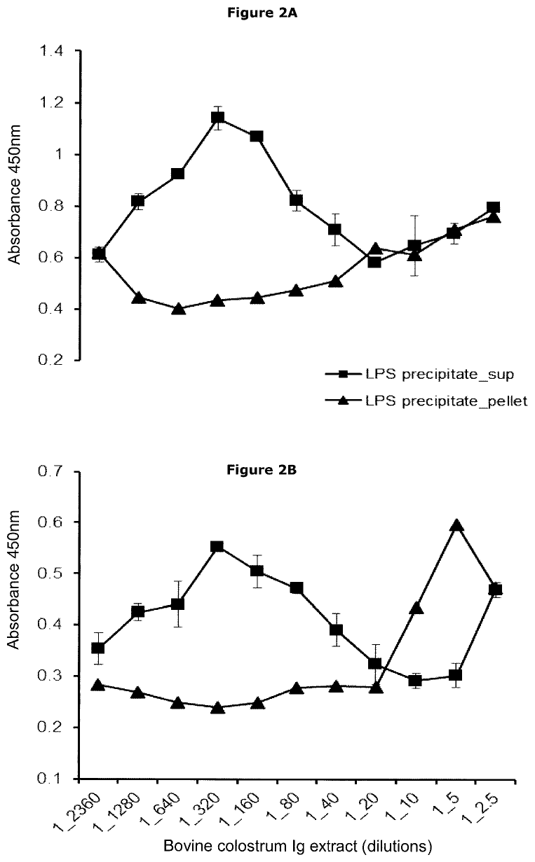

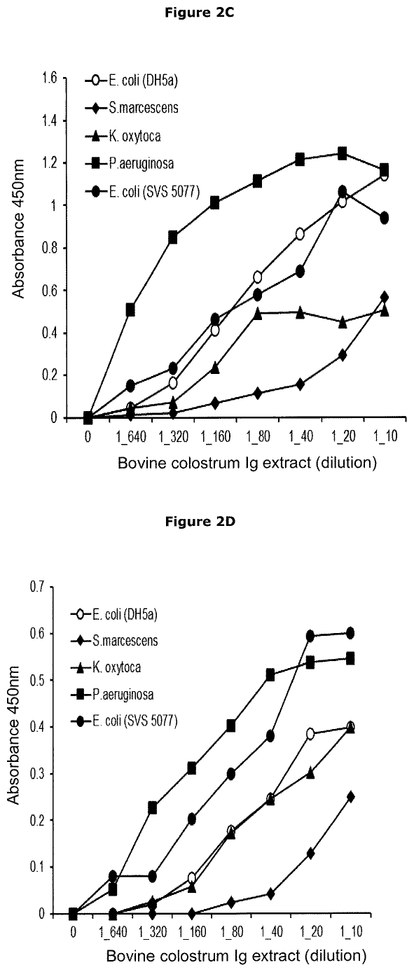

The precipitate and the supernatant were then assessed to determine whether LPS/IgG and/or LPS/IgA complexes were present. As shown in FIG. 2, both LPS/IgG complexes (FIG. 2A, FIG. 2C) and LPS/IgA complexes (FIG. 2B, FIG. 2D) were present in the precipitate, while the supernatant showed a decrease in LPS. This clearly demonstrated that immunoglobulins present in the colostrum preparation were able to form complexes with LPS and that these complexes precipitated out of solution. This was shown for E. coli LPS (FIG. 2A, FIG. 2B), and the selected gram-negative mastitis pathogens Serratia marcescens, Klebsiella oxytoca, Pseudomonas aeruginosa, in addition to two E. coli strains (see FIG. 2C, FIG. 2D).

Furthermore, as an immobilised polymyxin B (PMB) matrix was used in the ELISA to detect LPS, these data also show that despite the LPS being present in a complex, the Lipid A/KDO core region of the LPS was still capable for binding to polymyxin B.

The endotoxicity properties the various LPS/Ig precipitation fractions were then assessed, by testing both the precipitates and the supernatants in a standard functional Limulus Amoebecyte Lysate (LAL) assay.

As shown in FIG. 3A, LPS-dependent endotoxicity activity of the supernatant declined with increasing IgG concentration up to the 1:20 IgG dilution. Surprisingly, LPS present in the precipitate exhibited no endotoxin activity, as can be seen in FIG. 3B.

These data establish that colostrum immunoglobulins can not only bind to and precipitate LPS, but are also able to reduce endotoxicity associated with LPS.

Experiments to determine if IgG, in the absence of IgA, is capable of forming LPS/IgG complexes that reduce LPS endotoxicity were then performed. These experiments were considered because, without wishing to be bound by any theory, commercially available bovine colostrum or serum preparations generally contain only immunoglobulins of the IgG subclass as IgA is usually inactivated during the manufacturing process.

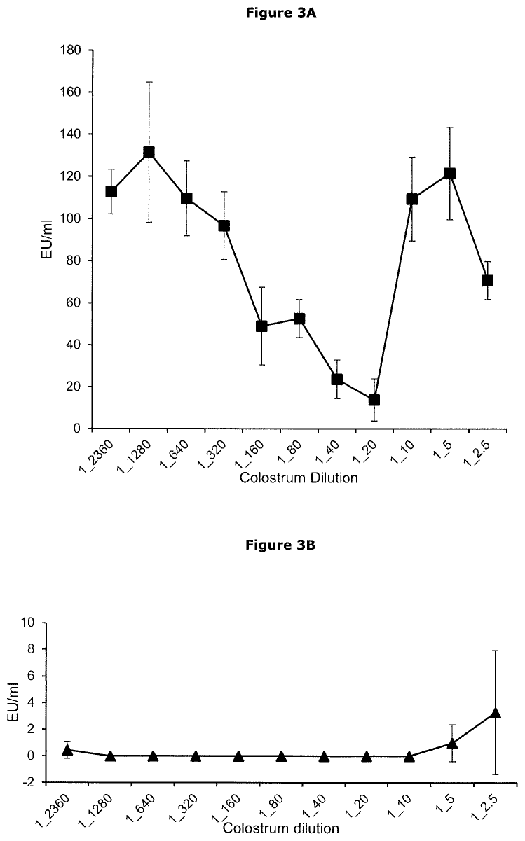

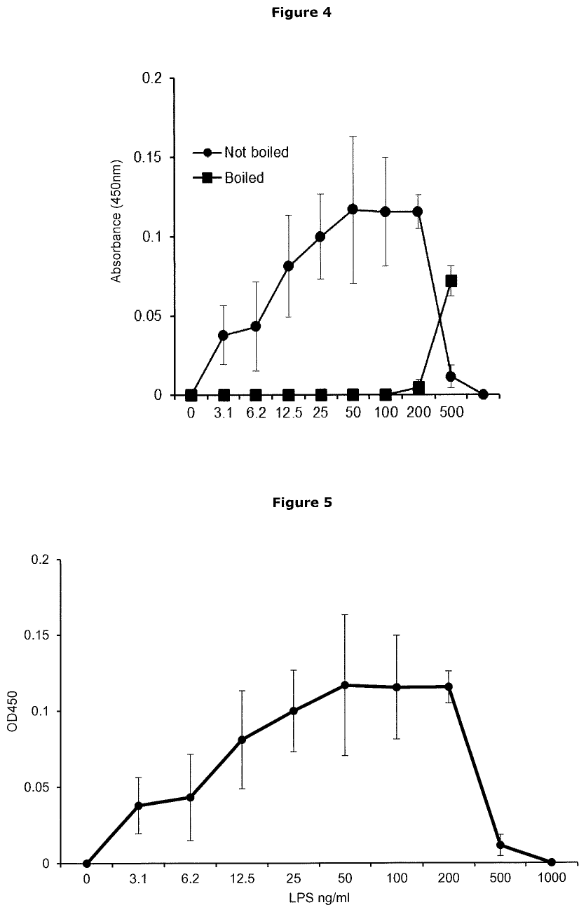

Polyclonal IgG (22 μg/ml) from a bovine colostrum IgG preparation was able to precipitate LPS over a range of LPS concentrations when no IgA was present, as demonstrated by protein analysis (see FIG. 4). Denaturation of LPS-protein complexes by boiling markedly reduced the amount of LPS-protein complexes present (FIG. 4, boiled).

As discussed above, a proportion of the precipitated LPS/IgG retained the ability to bind to PMB (see FIG. 5). This indicates that IgG binding to LPS does not preclude PMB binding to and/or PMB-based capture of the Lipid A/KDO core region of LPS, and in this case subsequent detection in the LPS ELISA.

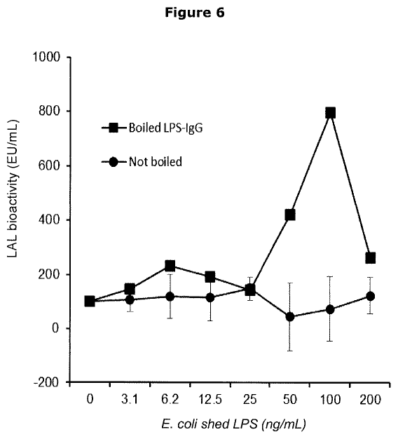

Notwithstanding the fact that LPS is capable of binding to PMB despite being bound to IgG, a significant decrease in LPS endotoxicity was found at concentrations of 50 and 100 ng/mL LPS (see FIG. 6). Denaturation of LPS-protein complexes by boiling markedly increased LAL bioactivity, and thus endotoxicity, of these preparations (FIG. 6, boiled vs not boiled).

Notably, these data show that the IgG-mediated reduction in endotoxicity and reduction in free LPS could be reversed by removal of IgG from LPS by heat denaturing the sample (FIG. 4 boiled, FIG. 6 boiled). This shows that IgG indeed can inhibit functionally important properties of LPS, such as endotoxicity.

### Conclusion

Taken together this data shows that bovine colostrum IgG and IgA antibodies can bind to free LPS to form LPS/IgG and LPS/IgA complexes that reduce LPS endotoxin activity (measured by LAL assay). The combination of bovine colostrum antibodies from the IgA and IgG subclass was found to be superior to the use of colostrum IgG alone.

### Example 2: Comparison of Bovine Colostrum Immunoglobulins and Other Immunoglobulins

This example presents experiments comparing the efficacy of bovine colostrum immunoglobulins in reducing LPS endotoxicity associated with gram-negative bacterial infections to immunoglobulins from other sources.

### Methods and Materials

Colostrum (Peptide Ignition, US) was used for this experiment as a source of IgG. Sample 168, an in-house verified acute E. coli mastitis milk sample obtained locally was used as the source of acute mastitis IgG.

LPS endotoxicity experiments were conducted as described above in Example 1 using a commercially available LAL assay.

### Results

In order to ascertain if IgG from different sources was effectively and/or functionally equivalent, IgG isolated from colostrum and IgG isolated from acute mastitis milk samples were assessed as described in Example 1 above.

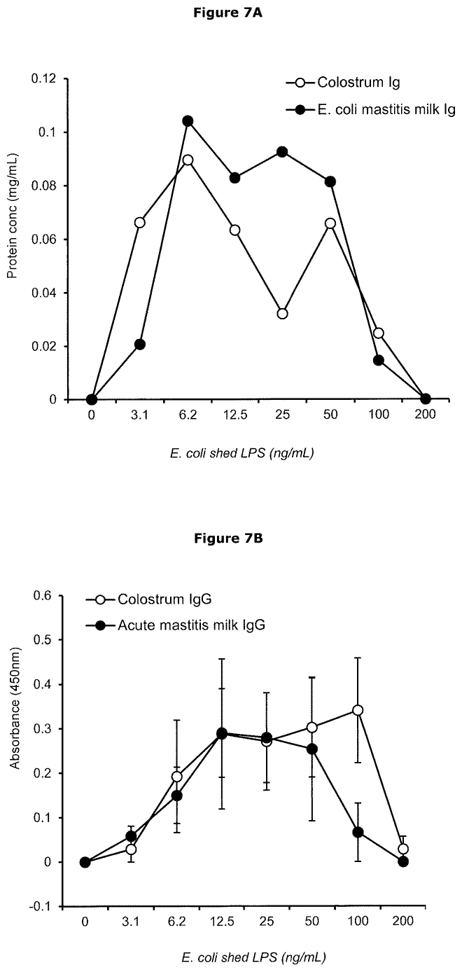

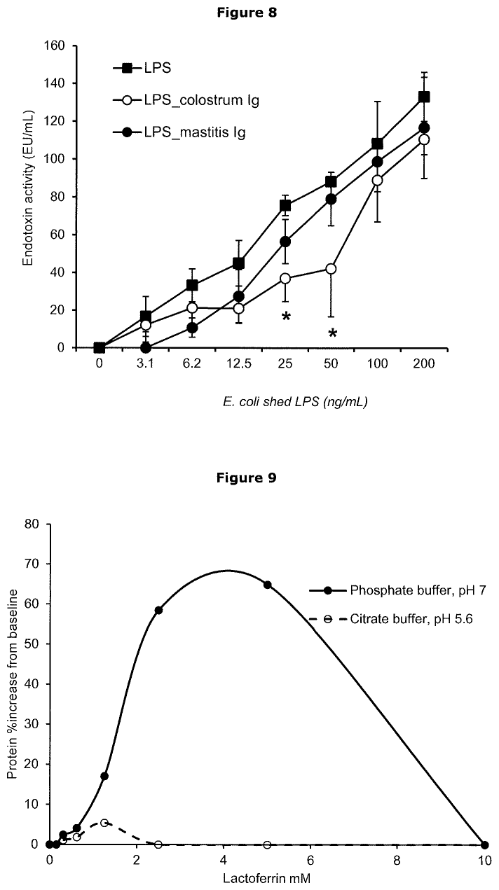

Notably, IgG from acute mastitis milk samples was significantly better (P<0.05) that IgG from colostrum in precipitating LPS (see FIG. 7). However, efficacy in precipitation did not correlate to efficacy in decreasing endotoxicity. As can be seen in FIG. 8, the reduction in the endotoxin activity of the supernatant was highly significantly better (P<0.001) for colostrum IgG than for IgG obtained from the mastitis milk sample.

### Conclusion

These data clearly show that IgG from colostrum and IgG from acute mastitis milk samples have different functional properties, and that colostrum IgG is superior in reducing the endotoxic activity of LPS.

### Discussion

Taken together, the results of the experiments presented in Examples 1 and 2 show that a polyclonal antibody pool obtained from bovine colostrum can bind and precipitate LPS to reduce endotoxicity. Further experiments established that colostrum Ig forms LPS/Ig complexes where, without wishing to be bound by any theory, the inventors expect that the bound antibodies mask the LPS region responsible for endotoxicity, reducing or ablating its activity. This in turn demonstrates that colostrum antibodies have novel functional properties which, again without wishing to be bound by any theory, support their use in therapy, for example to reduce or neutralise the disease-causing agents of endotoxin mastitis.

A combination of IgG and IgA from bovine colostrum demonstrated superior immunoprecipitation properties to IgG alone, although colostrum IgG alone was effective in providing a neutralising function.

Surprisingly, despite being somewhat less effective than IgG from acute mastitis milk samples at precipitating LPS from solution, IgG from colostrum was significantly better in removing the endotoxin activity from the supernatant. This differential activity was not expected, and suggests, without wishing to be bound by any theory, that IgG from differing sources has differing activity and that the ability of immunoglobulins to mediate a reduction in LPS endotoxicity is not necessarily correlated with efficacy in LPS-binding and/or complexation.

### Example 3: Assessment of Concanavalin a as a Therapeutic Agent

This example presents experiments assessing the efficacy of the plant lectin Concanavalin A (ConA), either alone or in the presence of bovine lactoferrin, in reducing LPS endotoxicity associated with gram-negative bacterial infections. The ability of ConA to aggregate the bacteria themselves was also assessed.

### Methods and Materials

ConA was obtained from a commercial source (Medicago, Sweden). ConA was reconstituted in Citrate Buffer pH5.4 and stored frozen until used in experiments.

Lactoferrin was obtained from Westland Milk Products (Hokitiko New Zealand). Lactoferrin was used at a concentration 100 μg/ml.

E. coli LPS was obtained from a well characterised E. coli strain 5077 originally isolated in house from an acute mastitis milk sample.

LPS precipitation experiments were conducted with various concentrations of ConA, ranging from 0.1 to 1 mg/ml.

Experiments were conducted by incubation of ConA and lactoferrin with the sample at 37° C. at a pH of 7.4 for 1 hour.

### Results

Initial experiments were designed to assess a therapeutic approach that utilises the pH dependent transformation of ConA from a dimeric to a tetrameric form. The highly glycosylated bovine protein lactoferrin (LF) was chosen as a suitable glycoprotein target. Bovine LF possesses a large number of mannose residues (compared to human lactoferrin and lactoferrins from other animals) on the surface that bind to ConA. As used herein, bovine LF represents a useful model antigen.

Initial experiments evaluated a specific concentration of ConA (10 mM) on a dilution of LF to demonstrate ConA binding upon pH-induced structural transformation.

As shown in FIG. 9, ConA precipitated the model antigen LF out of solution in a pH dependent manner. Little or no precipitation was observed at an acid pH of 5.4, but precipitation can clearly be detected at a pH of 7.0 (FIG. 9), consistent with a pH-dependent ConA dimer to tetramer transformation and transition from a soluble to an insoluble form.

The ability of ConA to bind and aggregate gram-negative bacteria derived LPS to neutralise endotoxicity was then assessed. Samples comprising E. coli were incubated in the presence or absence of ConA. The pH-dependent transition from a dimeric to a tetrameric form of ConA was initiated, and samples were then centrifuged. The level of LPS shed by E. coli in the samples was then measured by ELISA.

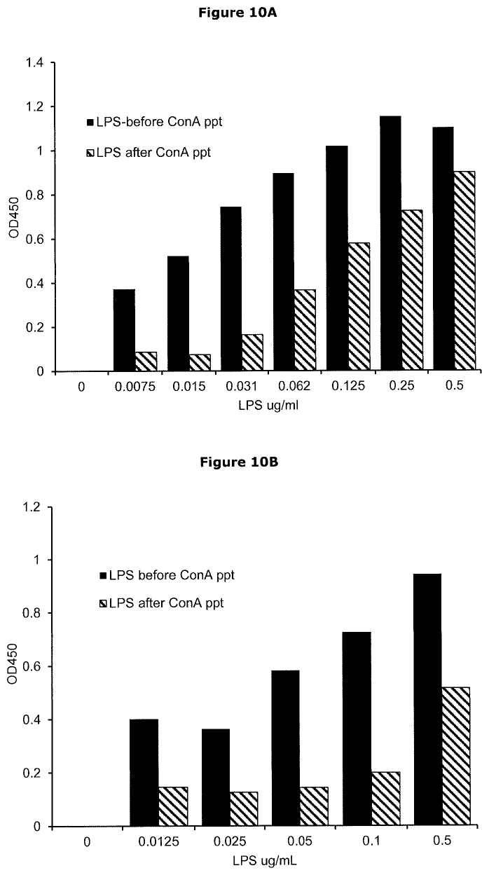

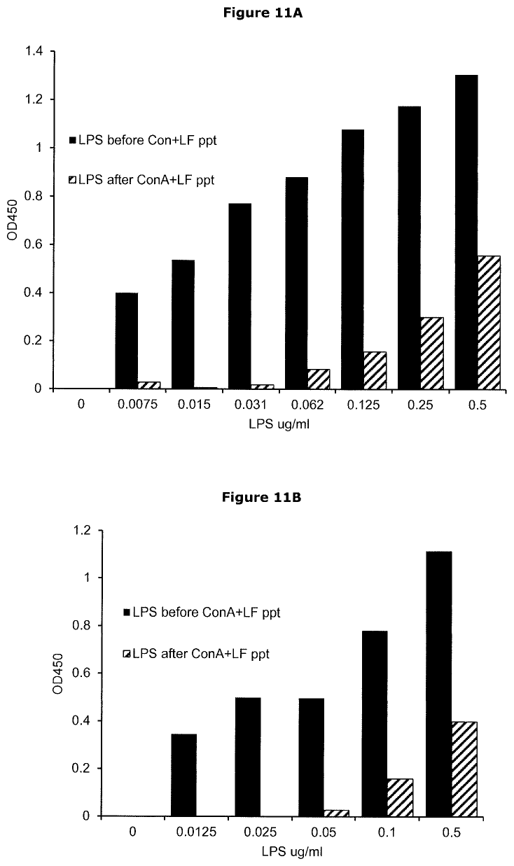

As shown in FIG. 10A, the amount of LPS remaining in the supernatant of samples incubated with ConA was substantially reduced after ConA precipitation and centrifugation.

Subsequent experiments showed that LPS from other gram-negative bacteria was also able to be removed from solution in the presence of ConA over a wide range of concentrations, again by promoting the transition of ConA from a dimeric to a tetrameric form. See FIG. 10B, which presents data for ConA-dependent precipitation of LPS from the mastitis pathogen Serratia marcescens.

Unexpectedly, ConA-dependent precipitation was enhanced over a wider range of LPS concentrations by the addition of LF, as shown in FIG. 11. The addition of LF statistically significantly improved the precipitation of both LPS from E. coli (FIG. 11A) and LPS from S. marcenscens (FIG. 11B).

The amount of LF present was also determined to influence ConA-dependent aggregation and precipitation of LPS. Table 1 below shows that ConA aggregation and precipitation of E. coli LPS was more effective when 0.1 mg/mL, rather than 0.05 mg/mL, of LF was present.

TABLE 1
Addition of LF facilitates ConA aggregation
and precipitation of E. coli LPS.

% LPS reduction by-ConA or ConA + LF
precipitation LF (mg/mL)

| LPS (μg/ml) | 0 | 0.1 mg/mL | 0.05 mg/mL |
|---|---|---|---|
| 0 | 0 | 0 | 0 |
| 0.0075 | 77.1 | 93.0 | 100 |
| 0.015 | 85.9 | 98.9 | 88.7 |
| 0.031 | 77.9 | 97.7 | 83.0 |
| 0.062 | 59.1 | 90.6 | 74.3 |
| 0.125 | 43.3 | 85.5 | 66.5 |
| 0.25 | 37.0 | 74.5 | 59.0 |
| 0.5 | 18.3 | 57.3 | 45.4 |

As shown in Table 1, at an LPS concentration of 0.125 μg/mL, the use of ConA alone resulted in a 43% reduction, whereas addition of 0.05 mg/mL and 0.1 mg/mL LF reduced the detection of LPS in the supernatant by 66.5% and 85.5%, respectively.

Taken together, the results shown in FIGS. 10 and 11 and Table 1 demonstrate that LPS derived from different gram-negative mastitis pathogens can be precipitated out of solution by employing pH dependent dimer to tetramer transformation of ConA. Furthermore, LPS precipitation is enhanced by the addition of the mannosylated glycoprotein LF.

### Clinical Samples

The efficacy of ConA (in the presence or absence of LF) in removing LPS present in clinical samples was then assessed.

Two independent sets of milk samples from cows suffering from mastitis associated with well characterised clinical coliform bacteria were obtained. The first set of samples was collected from a herd with a history of post-partum clinical acute mastitis. The second set of samples was obtained from a veterinary diagnostic lab and consisted of routine diagnostic acute mastitis milk samples. In both sets of samples, infection with gram-negative bacteria, and the presence of LPS/IgG complexes detected in the milk, were both verified.

These samples were assayed for ConA (with or without LF) aggregation and precipitation, as described above. The effectiveness of LPS removal from the clinical milk samples was then verified by measuring the endotoxicity using a commercial LAL assay.

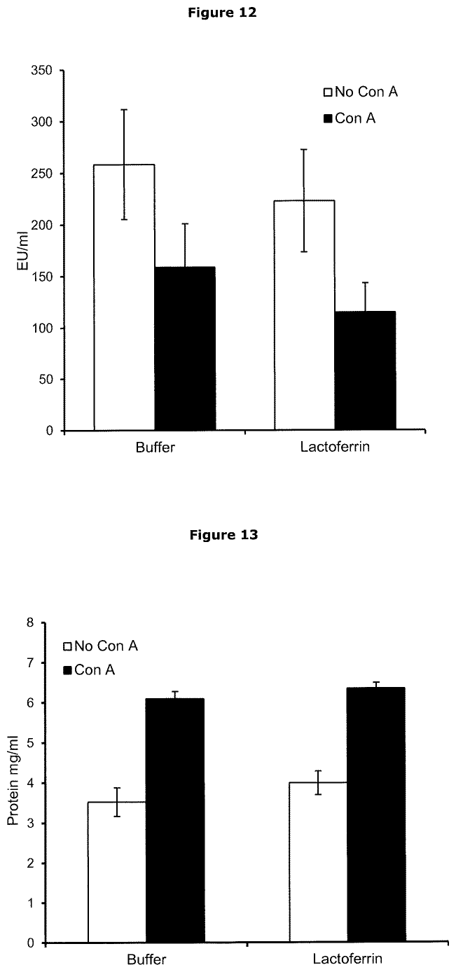

As can be seen in FIG. 12, ConA treatment was highly effective, as demonstrated by a significant (P<0.05) reduction of endotoxin activity in the samples (FIG. 12).

In these milk samples, a significant increase in the amount of protein precipitated by ConA was observed in the presence of LF (see FIG. 13). This data, in combination with the removal of endotoxin activity from the samples, strongly supports the efficacy of ConA, optionally together with LF, in the binding, aggregation, and precipitation of LPS present in clinical milk samples.

### Removal of Gram-Negative Bacteria

The milk samples collected from clinical acute mastitis cows discussed above were then used to determine the effectiveness of ConA (with or without LF) to bind to and aggregate gram-negative bacteria (as evidenced by clumping) present in the samples.

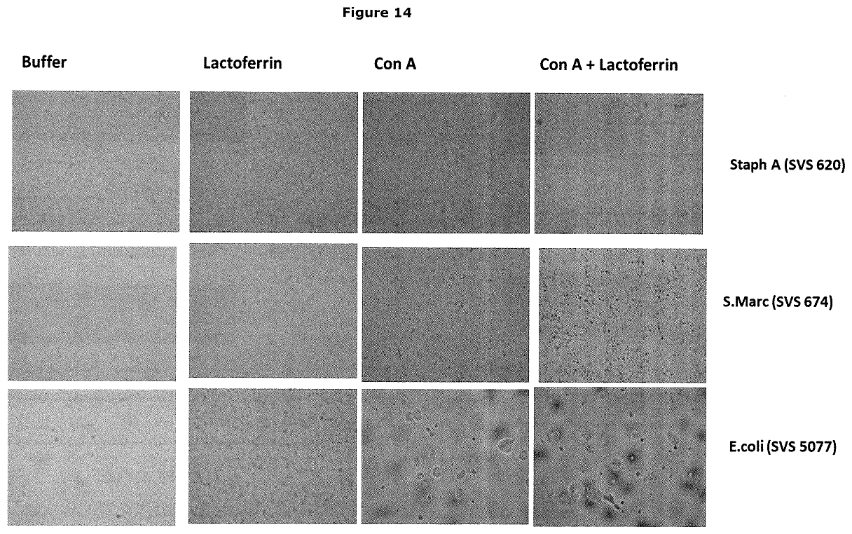

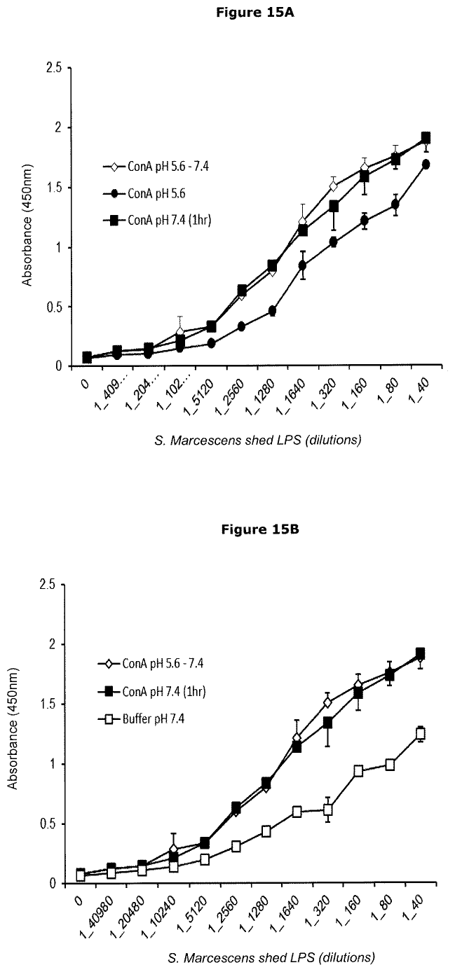

As shown in FIG. 14, incubation of the milk samples with ConA, and with ConA in the presence of LF, was effective to cause clumping on live gram-negative bacteria present in the samples. Clumping with ConA, and with ConA+LF, was observed for two gram-negative mastitis pathogens, Serratia marcescens (FIG. 14, middle row), and E. coli (FIG. 14, bottom row). Additional experiments established that treatment of bacteria with ConA and LF did not induce LPS shedding (data not shown). Notably, no clumping was observed with the gram-positive bacteria, Staphylococcus aureus, present in the sample, (FIG. 14, top row).

### Discussion

The data presented in this example demonstrates that ConA, either alone or in the presence of bovine lactoferrin, effectively binds, aggregates, and neutralises shed LPS. Furthermore, the data presented in this example demonstrates ConA is effective in removing LPS at a physiologically relevant pH (pH 7.4). Without wishing to be bound by any theory, the inventors believe this to be due to the pH-mediated dimer to tetramer transformation ConA undergoes at this pH.

The application of ConA-mediated LPS removal to clinical acute coliform mastitis cases showed an increase in protein aggregation and precipitation resulting in an overall reduction in milk endotoxin activity.

Surprisingly, addition of ConA, and ConA together with LF, to samples comprising clinically relevant live gram-negative bacteria caused bacterial clumping with no increase in LPS shedding, which supports a protective role of ConA and lactoferrin. This was determined by detecting LPS in an ELISA, based on the ability of LPS to bind to polymyxin B coating the wells, followed by labelling with a colostum antibody-based detection system.

### Conclusion

Here, ConA (either alone or in combination with lactoferrin) has been shown to (i) bind and precipitate LPS (free LPS and LPS present in complexes with other molecules present in the biological samples, such as endogenous antibodies, LPS binding protein, or soluble CD1) shed from gram-negative bacteria in laboratory samples and in clinical milk samples (ii) reduce overall endotoxicity again in laboratory samples and in clinical milk samples, and (iii) specifically bind to and aggregate (clump) gram-negative bacteria present in clinical milk samples.

These experiments clearly show the efficacy and therapeutic potential of ConA, optionally in combination with bovine lactoferrin, to reduce overall endotoxicity and promote gram-negative bacteria clearance, for example from the mammary gland in subjects with mastitis.

### Example 4: Assessment of Concanavalin a Binding to LPS

This example presents experiments investigating the impact of the multimeric state of concanavalin on its ability to bind to and aggregate LPS. In particular, these experiments investigated the effect of the pH driven transformation of concanavalin A from a dimeric to a tetrameric form on concanavalin's ability to aggregate LPS.

### Methods and Materials

The influence of raising pH on the ability of concanavalin A to enhance the detection of LPS in an ELISA was assessed. Various concentrations of LPS from S. marcescens were prepared and bound to polymyxin B matrix as described above. Buffer pH was raised from pH 5.6 to 7.4 during incubation of the polymyxin B matrix-bound LPS with colostrum IgG extract.

### Results

As shown in FIG. 15, the pH buffer shift from pH 5.6 to 7.4 during the incubation of LPS with colostrum IgG enhanced the binding of IgG to LPS, raising binding from that observed at pH 5.6 throughout (FIG. 15A) to a level comparable to that observed when the incubation was carried out at pH 7.4 from the outset (FIG. 15A, 15B).

### Discussion

The data presented in this Example shows that Con A can bind to LPS. Without wishing to be bound by any theory, the inventors believe that Con A binds to glycoproteins on the LPS and the transformation from dimer to tetramer may create new epitope regions for colostrum IgG to bind to and thus enhance the detection of LPS. These data support the utility of this transformation of Con A in the aggregation and removal of gram-negative bacteria via LPS binding in clinical scenarios, for example, in the identification and treatment of bacterial infections such as intramammary coliform mastitis.

### Example 5: Transformation of Concanavalin a Enhances Binding to Selective Gram-Negative Bacteria LPS

This example presents experiments investigating the binding of derivatised Con A to LPS derived from various gram-negative bacteria. Here, the binding of biotinylated Con A during the pH-driven transformation from dimeric to tetrameric Con A was examined.

### Methods and Materials

Biotinylated Con A dimers were stored in a pH 5.4 buffer prior to incubation with polymyxin B matrix bound LPS shed by the gram-negative bacteria E. coli, S. marcescens, P. aeruginosa, E. cloacae, and K. oxytoca. Binding was determined by ELISA as described above.

### Results

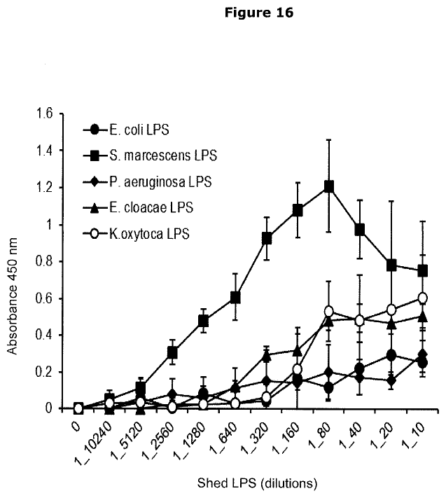

As shown in FIG. 16, biotinylated Con A bound with varying affinities to LPS from different gram-negative bacteria. Biotinylated Con A showed greatest binding to LPS shed from S. marcescens, whereas weaker binding was observed with LPS shed from a bovine mastitis E. coli strain, E. coli SVS-500).

### Discussion

The data presented in this Example indicates that derivatisation of Con A does not negatively affect its ability to bind to LPS. Furthermore, biotinylated Con A showed varying affinities for LPS from different gram-negative bacteria, including during the pH-driven transformation from dimers to tetrameric complexes.

### Example 6: Transformation of Concanavalin a Enhances the Detection of Selective Gram-Negative Bacterial LPS

This example presents experiments investigating the binding of Con A to LPS derived from various gram-negative bacteria. Here, these experiments investigated the effect of the pH driven transformation of concanavalin A from a dimeric to a tetrameric form on its ability to aggregate LPS.

### Methods and Materials

LPS from the gram-negative bacteria E. coli, K. oxytoca, P. aeruginosa, and S. marcescens was prepared and various concentrations were bound to polymyxin B matrix as described above. Con A at pH 5.4 was then added and incubated, followed by quantification of binding by ELISA as described above.

### Results

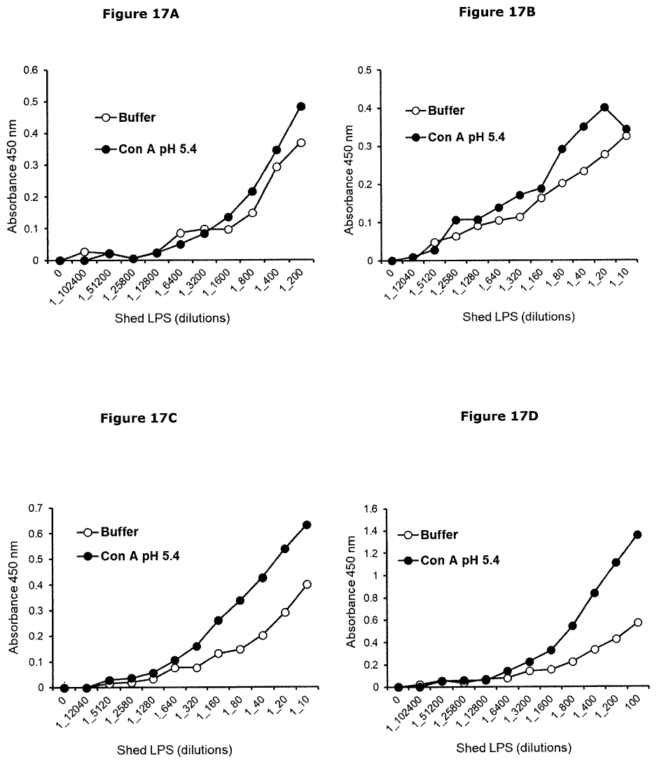

As shown in FIG. 17, the pH-dependent transformation Con A enhanced the detection of LPS shed from P. aeruginosa (see FIG. 17C) and S. marcescens (FIG. 17D). No pH-dependent enhancement of LPS binding by Con A was observed with LPS shed from E. coli (FIG. 17A) or K. oxytoca (FIG. 17B) bacteria.

### Discussion

The data presented in this Example shows that the transformation of Con A dimers to tetramers enables the binding to LPS derived from certain gram-negative bacteria, and enhanced the detection pf such LPS by ELISA. Notably, enhanced binding and detection was not observed with LPS shed from other gram-negative bacteria in these experiments.

### Example 7: Transformation of Concanavalin a Binds to and Aggregates Bovine Lactoferrin

This example presents experiments assessing the influence of other agents on the aggregation of gram-negative bacteria during the multimeric transformation of Con A.

Here, Con A binding of bovine lactoferrin, and the impact of the transformation of Con A from one multimeric form to another on binding to bovine lactoferrin, was assessed.

### Methods and Materials

Varying concentrations of bovine lactoferrin (25 ng/mL to 400 ng/mL) were prepared. A preparation of Con A dimers (pH 5.4, 0.5 mg/mL) were then added and incubated. Samples were centrifuged as described above. Binding of Con A to and aggregation of bovine lactoferrin was measured by spectrophotometric analysis of precipitated protein, and separately by SDS-PAGE analysis of the supernatant of samples incubated with increasing amounts of Con A.

### Results

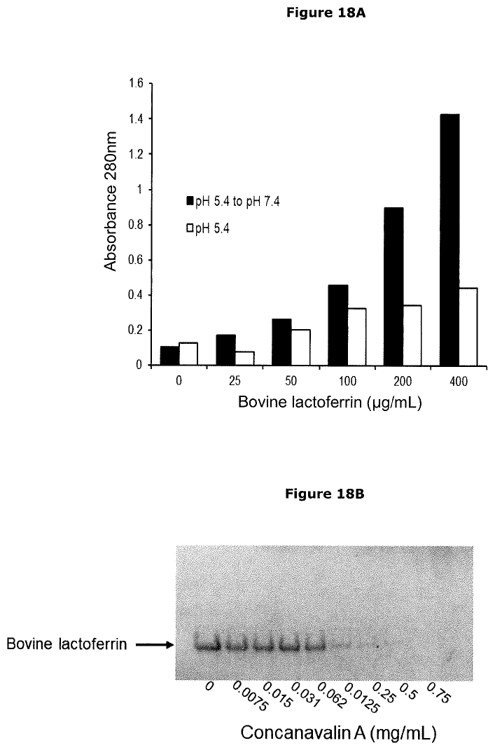

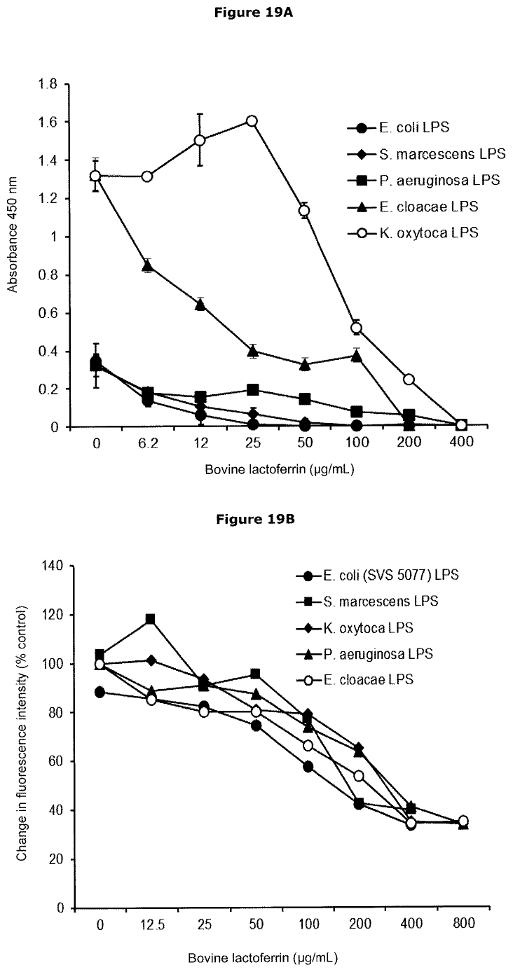

As shown in FIG. 18, using a fixed amount of Con A dimers a dose-dependent increase in precipitated protein was observed when the pH driven transformation of Con A dimers to tetramers was carried out. In contrast, no lactoferrin aggregation was observed if an acidic buffer (pH 5.4) was used (see FIG. 18A).

As can be seen in FIG. 18B, increasing dose of Con A dimers caused a dose-dependent increase in lactoferrin aggregation and increasing absence of lactoferrin from the supernatant.

### Discussion

The data presented in this Example established that bovine lactoferrin can be bound to and aggregated during the pH driven transformation of Con A dimers to tetramers.

### Example 8: Bovine Lactoferrin Binding to Gram-Negative Bacteria LPS

This example presents experiments investigating the ability of bovine lactoferrin to bind to LPS from various gram-negative bacteria.

### Methods and Materials

Various concentrations of bovine lactoferrin (6.2 μg/mL to 400 μg/mL) were prepared and incubated for 1 h with LPS from the gram-negative bacteria E. coli, S. marcescens, P. aeruginosa, E. cloacae, and K. oxytoca. LPS binding was assessed using a polymyxin B/PEG-20 capture matrix by ELISA as herein described.

The impact of bovine lactoferrin on endotoxin activity was also determined by assessing LAL bioactivity.

### Results

As shown in FIG. 19A, incubation with bovine lactoferrin caused a dose-dependent reduction in the detection of LPS by ELISA. As shown in FIG. 19B, incubation with bovine lactoferrin also showed a dose-dependent decrease in endotoxin activity.

### Discussion

The data presented in this Example established that bovine lactoferrin is able to bind to and sequester LPS from each of the gram-negative bacteria tested. Without wishing to be bound by any theory, the inventors believe that lactoferrin has utility in combination with Con A, and/or with the multimeric transformation of Con A, to aggregate and remove gram-negative bacteria.

### Example 9: Bovine Lactoferrin Binding to Gram-Negative Bacteria LPS

This example presents experiments assessing the efficacy of a combination of ConA and bovine lactoferrin in binding to and aggregating LPS derived from a various gram-negative bacteria, and in reducing LPS endotoxicity associated with gram-negative bacterial infections. The ability of the combination to aggregate the bacteria themselves was also assessed.

### Methods and Materials

ConA and lactoferrin were obtained and prepared as described in Example 3 above. The pH-dependent multimeric transformation of Con A was carried out as described above in Examples 3 and 4. Binding to and aggregation of bacterial samples was carried out as described above in Example 3.

### Results

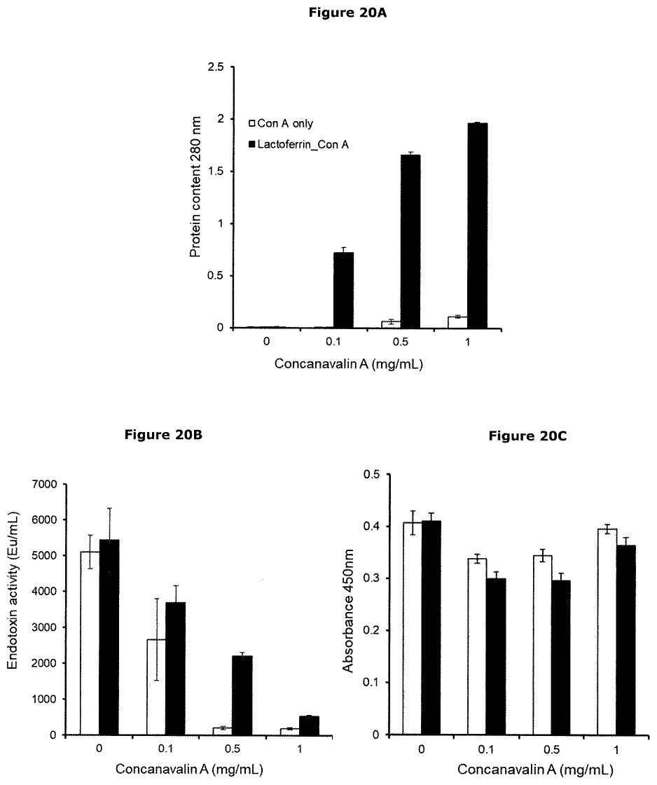

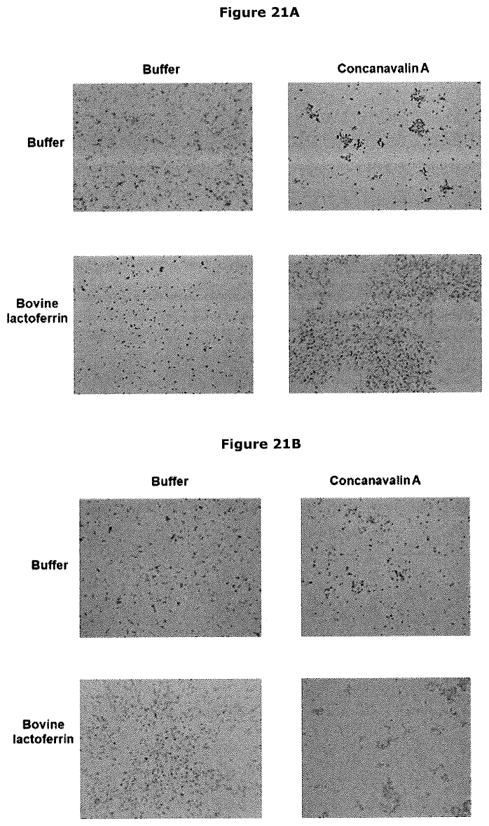

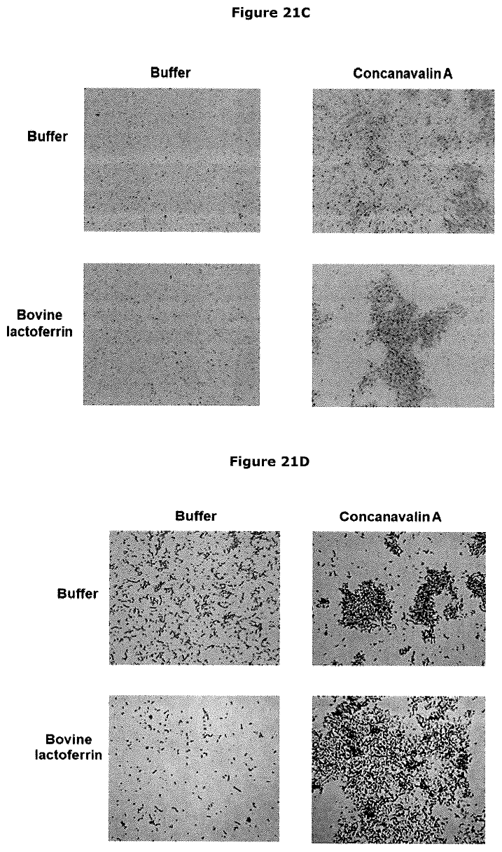

As shown in FIG. 20, lactoferrin bound to LPS followed by pH-dependent ConA transformation effectively sequestered LPS out of solution (see FIG. 20A, 20C), removing endotoxin activity (FIG. 20B) from the supernatant and reducing LPS levels in the supernatant (FIG. 20C). As shown in FIG. 21, incubation of the bacteria with a combination of bovine lactoferrin and Con A caused increased aggregation of the gram-negative bacteria K. oxytoca (FIG. 21A), E. coli (FIG. 21B), S. marcescens (FIG. 21C), and P. aeruginosa (FIG. 21D) when compared to lactoferrin alone and Con A alone. This was particularly evident with S. marcescens and P. aeruginosa.

### Discussion

The data presented in this Example established that a combination of bovine lactoferrin and Con A was markedly effective in binding to and sequestering LPS from each of the gram-negative bacteria tested. Furthermore, this combination was effective in reducing endotoxin activity associated with LPS from gram-negative bacteria, and particularly effective in aggregating the bacteria themselves. Without wishing to be bound by any theory, the inventors believe that a combination of lactoferrin and Con A, optionally employed together with the multimeric transformation of Con A, thus has efficacy in mitigating the effects of gram-negative bacterial infections and endotoxicity associated therewith.

### Example 10: Bovine Colostrum Ig Extract Containing IgG and IgA Agglutinates Gram-Negative Bacteria

This example presents experiments assessing the binding to and agglutination of gram-negative bacteria with Ig present in pooled bovine colostrum Ig extracts, and with bovine colostrum IgG extracts.

### Methods and Materials

E. coli (SVS 5077) as resuspended in PBS, pH 7.4 and adjusted to 1×10⁹ bacteria/mL.

### Bovine Colostrum Preparation:

(a) Ig extract (consisting of IgG and IgA)
(b) IgG extract

Varying amounts of E. coli (0.25-2×10⁸) were mixed with a fixed concentration of colostrum Ig or IgG extract (0.5 mg/mL) and incubated for 1 h at 37° C., followed by o/n at 4° C. Bacteria samples were placed on a glass slide and viewed under a light microscope (100× magnification) to detect clumping (i.e., agglutination).

Additionally, gram-negative bacteria K. oxytoca, P. aeruginosa, and E. coli strains SVS 5077 and Dh5a (1.5×10⁸) were incubated with (a) buffer, or 0.5 mg/mL of (b) IgG or (c) Ig (IgG/IgA) extracts from bovine pooled colostrum for 1 hr at 37° C. followed by o/n at 4° C. Bacterial samples were then placed on glass slides and viewed using a light microscope at 400× magnification.

### Results

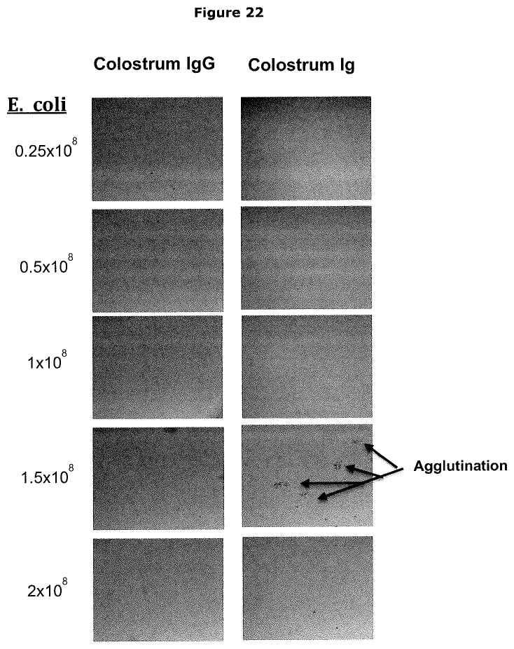

As shown in FIG. 22, agglutination was only observed when 1.5×10⁸ E. coli was incubated with Bovine pooled colostrum Ig extract that contained both IgG and IgA. No clumping was observed when the same bacteria number (1.5×10⁸) was incubated with an IgG extract of the same bovine pooled colostrum.

Only a bovine colostrum Ig extract containing both IgG and IgA agglutinated with E. coli bacteria. Without wishing to be bound by any theory, this suggests to the inventors that IgA may be responsible for promoting the agglutination of E. coli bacteria.

These parameters were then used to explore the ability of bovine pooled colostrum Ig and IgG extracts to agglutinate different gram-negative bacteria responsible for clinical mastitis in cows.

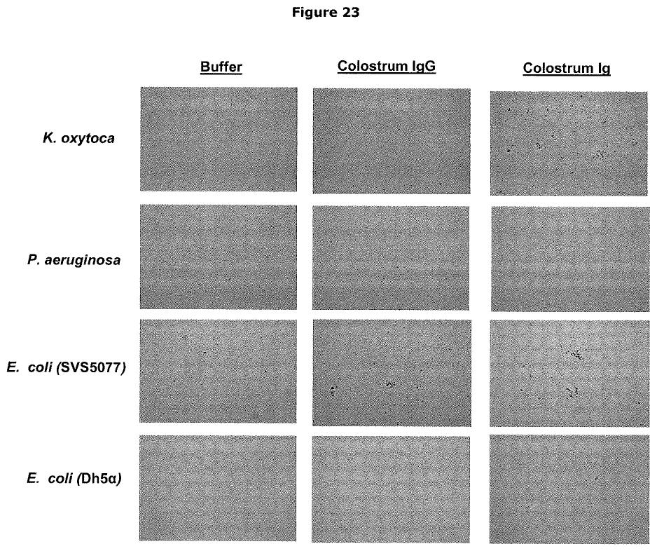

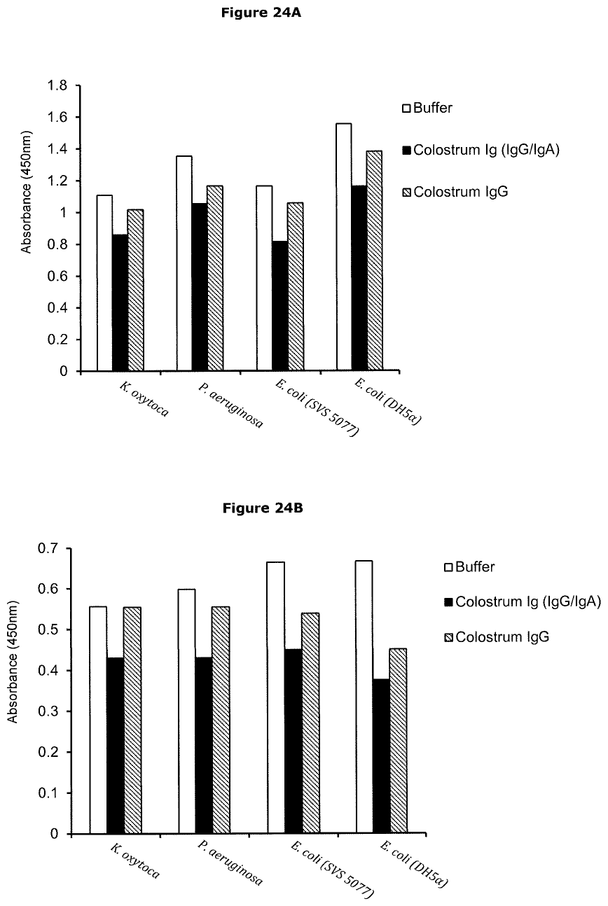

As shown in FIG. 23, bacteria incubated with PBS buffer only showed no or only minimal clumping. Incubation with colostrum IgG extract also caused no or minimal clumping of bacteria, although some agglutination was evident in P. aeruginosa samples. However, agglutination was evident in all bacteria tested after incubation with an Ig colostrum extract containing both IgG and IgA.

### Discussion

The data presented in this Example provide further support establishing that an Ig extract of bovine pooled colostrum enriched in both IgG and IgA (in the absence of other milk proteins) can agglutinate gram-negative bacteria that cause clinical mastitis in cows.

### Example 11: Reduction of Gram-Negative Bacteria LPS after Agglutination with Bovine Colostrum Ig Extracts

This example presents experiments further assessing binding to and agglutination of gram-negative bacteria with bovine colostrum preparations and any concomitant reduction in detection of LPS.

### Methods and Materials

Gram-negative bacteria K. oxytoca, P. aeruginosa, and E. coli strains SVS 5077 and Dh5a were incubated with (a) buffer (negative control), (b) 0.5 mg/mL colostrum Ig (IgG/IgA) extract, or (c) colostrum IgG extract, prepared as described in Example 9 above. Samples were then centrifuged for 0.5×g for 1 min. The supernatant was collected and assessed for the presence of LPS using ELISA and detection system using anti-IgG and anti-IgA as described in the above Examples.

### Results

As shown in FIG. 24, incubation of all the tested gram-negative bacteria with bovine pooled colostrum Ig (IgG/IgA) extract caused a reduction in the amount of LPS in the supernatant detected (using either anti-IgA or anti-IgG) when compared to the negative control (incubation with buffer only).

A modest reduction in LPS detection was observed when P. aeruginosa, E. coli (SVS5077) and E. coli (DH5a) bacteria were incubated with an IgG extract. T

### Discussion

The data presented in this Example shows that bovine pooled colostrum Ig (IgG/IgA) reduced the amount of free LPS from gram-negative bacteria, in addition to LPS attached to the bacteria. Without wishing to be bound by any theory, the inventors believe these data support the efficacy of bovine colostrum Ig extracts in reducing the amount of gram-negative bacterial LPS that is capable of evoking an inflammatory response, such as in the mammary gland, and thus support the efficacy of such extracts in the treatment and/or prevention of diseases or conditions caused by or associated with gram-negative bacteria, and particularly those associated with endotoxicity and/or the presence of LPS.

### Example 12: Effect of Milk Ig Extracts on E. coli Shed LIPS Induced Pro-Oxidants in Milk Granulocytes

This example presents experiments assessing the ability of milk Ig extracts to modulate effectors of the host immune response to gram-negative bacterial LPS. More particularly, these experiments assessed the ability of colostrum or milk Ig extracts to modulate the levels of milk granulocyte pro-oxidants detected in response to E. coli shed LPS.

### Methods and Materials

Varying concentrations of E. coli shed LPS (12.5 ng/mL-200 ng/mL) were incubated with freshly isolated milk granulocytes for 20 mins. Pro-oxidant levels were measured over this timecourse using the intracellular fluorescent probe H₂DCFH.

Varying concentrations of E. coli shed LPS (3.1 ng/mL-200 ng/mL) were then preincubated with (a) colostrum Ig extract, and (b) E. coli mastitis milk Ig extract, prior to incubation with freshly isolated milk granulocytes. Pro-oxidant levels were again measured using H₂DCFH.

### Results

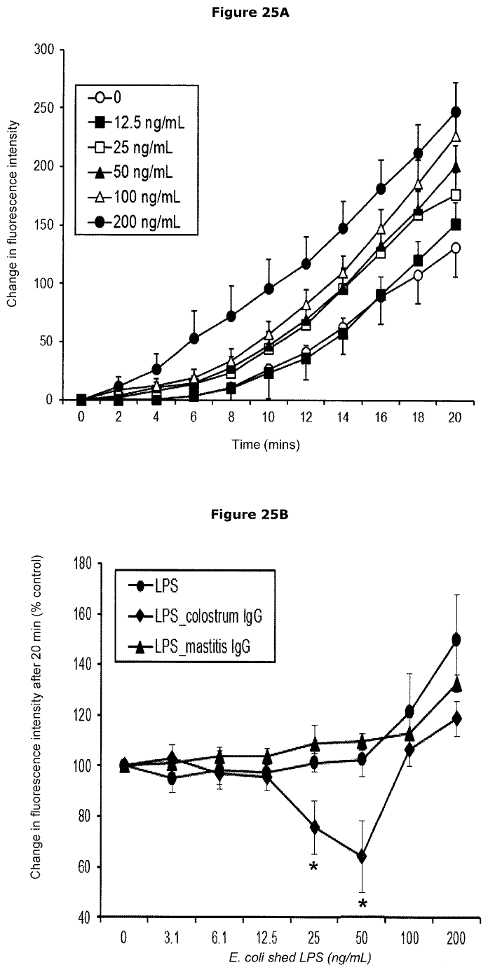

As shown in FIG. 25A, E. coli shed LPS caused a dose-dependent increase in pro-oxidants levels in milk granulocytes. As shown in FIG. 25B, preincubation of E. coli shed LPS with colostrum Ig extract suppressed pro-oxidant generation in a dose dependent manner. This suppression was not observed when an Ig extract of E. coli mastitis milk was used for the preincubation.

### Discussion

The data presented in this Example show bovine colostrum Ig binds to E. coli shed LPS preventing its ability to evoke pro-oxidants production and/or release in milk granulocytes. Mammary grand granulocytes have been reported to be instrumental in initiating and augmenting clinical mastitis. The inventors believe (without wishing to be bound by any theory) that the amelioration of LPS-induced pro-oxidants by colostrum Ig demonstrated in this Example may contribute to alleviate inflammation and tissue damage in diseases or conditions associated with LPS-induced immune responses, such as mastitis caused by gram-negative bacteria.

The entire disclosures of all applications, patents and publications cited above and below, if any, are herein incorporated by reference.

Where in the foregoing description reference has been made to integers or components having known equivalents thereof, those integers are herein incorporated as if individually set forth.

It should be noted that various changes and modifications to the presently preferred examples described herein will be apparent to those skilled in the art. Such changes and modifications may be made without departing from the spirit and scope of the invention and without diminishing its attendant advantages. It is therefore intended that such changes and modifications be included within the present invention.

The invention may also be said broadly to consist in the parts, elements and features referred to or indicated in the specification of the application, individually or collectively, in any or all combinations of two or more of said parts, elements or features.

Aspects of the invention have been described by way of example only, and it should be appreciated that variations, modifications, and additions may be made without departing from the scope of the invention, for example when present the invention as defined in the indicative claims. Furthermore, where known equivalents exist to specific features, such equivalents are incorporated as if specifically referred in this specification.

1. A method of treating or preventing a disease or condition caused by or associated with a bacterial infection in a subject in need thereof, the method comprising administering to the subject an effective amount of immunoglobulin, said immunoglobulin capable of binding specifically to one or more bacterial lipopolysaccharides, wherein the immunoglobulin comprises, consists essentially of, or consists of immunoglobulin in or from colostrum.

2. A method of treating or preventing a disease or condition caused by or associated with a bacterial infection in a subject in need thereof, the method comprising administering to the subject an effective amount a multimerizing reagent capable of binding to one or more lipopolysaccharides from gram-negative bacteria, wherein when bound to and/or complexed with said one or more lipopolysaccharides present in the subject said multimerizing reagent is capable of further multimerization.

3. A method of treating or preventing a bacterial infection in a subject in need thereof, the method comprising administering to the subject an effective amount of immunoglobulin, said immunoglobulin capable of binding specifically to one or more bacterial lipopolysaccharides, wherein the immunoglobulin comprises, consists essentially of, or consists of immunoglobulin in or from colostrum.

4. A method of treating or preventing a bacterial infection in a subject in need thereof, the method comprising administering to the subject an effective amount a multimerizing reagent capable of binding to one or more lipopolysaccharides from gram-negative bacteria, wherein when bound to and/or complexed with said one or more lipopolysaccharides present in the subject said multimerizing reagent is capable of further multimerization.

5. The method according to any one of the preceding claims wherein the bacterial infection is an infection of gram-negative bacteria.

6. The method according to any one of the preceding claims wherein the treatment or prevention comprises treatment, prevention, or amelioration of one or more symptoms of or associated with bacterial infection.

7. A method of treating or preventing mastitis in a subject in need thereof, the method comprising administering to the subject an effective amount of immunoglobulin, said immunoglobulin capable of binding specifically to one or more lipopolysaccharides from gram-negative bacteria, wherein the immunoglobulin comprises, consists essentially of, or consists of immunoglobulin in or from colostrum.

8. A method of treating or preventing mastitis in a subject in need thereof, the method comprising administering to the subject an effective amount of a multimerizing reagent capable of binding to one or more lipopolysaccharides from gram-negative bacteria, wherein when bound to and/or complexed with said one or more lipopolysaccharides present in the subject said multimerizing reagent is capable of further multimerization.

9. The method according to any one of the preceding claims wherein the treatment or prevention comprises treatment, prevention, or amelioration of one or more symptoms of or associated with mastitis.

10. The method according to any one of the preceding claims wherein the gram-negative bacteria is a bacteria selected from the group consisting of Escherichia spp., Pasteurella spp., Serratia spp., and Klebsiella spp.

11. The method according to any one of the preceding claims wherein the effective amount is an amount effective to bind to and/or reduce endotoxicity associated with said lipopolysaccharide.

12. The method according to any one of the preceding claims wherein the effective amount is an amount effective to form an insoluble complex comprising lipopolysaccharide.

13. The method according to any one of the preceding claims wherein the immunoglobulin comprises, consists essentially of, or consists of immunoglobulin from colostrum.

14. The method according to any one of the preceding claims wherein the immunoglobulin comprises, consists essentially of, or consists of IgG and IgA from colostrum.

15. The method according to claim 13 or 14, wherein the colostrum is bovine colostrum, including pooled bovine colostrum.

16. The method according to any one of the preceding claims wherein the multimerizing agent is selected from the group consisting of a lectin, a protein capable of binding lipopolysaccharide, and a lipopolysaccharide-binding compound.

17. The method according to any one of the preceding claims wherein the multimerizing agent is a lipopolysaccharide binding protein.

18. The method according to any one of the preceding claims wherein the multimerizing agent is Concanavalin A.

19. The method according to any one of the preceding claims wherein at least some of the Concanavalin A is dimeric, for example is dimeric when administered.

20. The method according to claim 18 or 19, wherein prior to administration at least some of the Concanavalin A is maintained at or below pH 5.4.

21. The method according to any one of the preceding claims, wherein the multimerizing agent is administered together with one or more agents selected from the group consisting of: a glycoprotein, a pharmaceutically acceptable non-ionic detergent, a pharmaceutically acceptable molecular crowding agent, and a pharmaceutically acceptable capture reagent.

22. The method according to any one of the preceding claims wherein the glycoprotein is lactoferrin, for example bovine lactoferrin.

23. The method according to any one of the preceding claims, wherein the method comprises administering to the subject an effective amount of Concanavalin A and lactoferrin.

24. The method according to any one of the preceding claims wherein the at least one capture reagent is selected from the group consisting of: a molecular crowding agent, a polyethylene, a polyethylene glycol, and Polymyxin B.

25. The method according to any one of the preceding claims, wherein administration to the subject comprises parenteral administration.

26. The method according to any one of the preceding claims, wherein administration to the subject comprises topical or oral administration, or administration to a mucosal tissue.

27. The method according to any one of the preceding claims, wherein administration to the subject comprises instillation, for example intra-mammary instillation.

28. The method according to any one of the preceding claims, wherein administration to the subject comprises administration ex vivo.

29. The method according to any one of the preceding claims, wherein ex vivo administration comprises administration to a sample from the subject, followed by administration of at least some of the sample to the subject.

30. A pharmaceutical composition comprising immunoglobulin capable of binding specifically to one or more bacterial lipopolysaccharides, wherein the immunoglobulin comprises, consists essentially of, or consists of immunoglobulin from colostrum.

31. The pharmaceutical composition according to claim 30, wherein the immunoglobulin comprises, consists essentially of, or consists of IgG and IgA from colostrum.

32. The pharmaceutical composition according to claim 30 or 31, wherein the colostrum is bovine colostrum, including pooled bovine colostrum.

33. A pharmaceutical composition comprising an effective amount a multimerizing reagent capable of binding to one or more lipopolysaccharides from gram-negative bacteria, wherein when bound to and/or complexed with said one or more lipopolysaccharides present in the subject said multimerizing reagent is capable of further multimerization.

34. The pharmaceutical composition according to claim 33 wherein the multimerizing agent is Concanavalin A.

35. The pharmaceutical composition according to claim 33 or 34, wherein the pharmaceutical composition comprises lactoferrin.

36. The pharmaceutical composition according to claim 35, wherein the lactoferrin is bovine lactoferrin.

37. The pharmaceutical composition according to any one of claims 33 to 36, wherein the pharmaceutical composition comprises one or more additional agents selected from the group consisting of: a pharmaceutically acceptable non-ionic detergent, a pharmaceutically acceptable molecular crowding agent, and a pharmaceutically acceptable capture reagent.

38. The pharmaceutical composition according to claim 37, wherein the capture reagent is selected from the group consisting of a lectin, a protein capable of binding lipopolysaccharide, and a lipopolysaccharide binding compound.

39. The pharmaceutical composition of any one of claims 30 to 38, for use in the treatment or prevention of a bacterial infection of gram-negative bacteria or of a disease or condition caused by or associated with a bacterial infection of gram-negative bacteria.

40. A pharmaceutical composition for reducing the amount of one or more lipopolysaccharides present in a subject or a sample from a subject, wherein the pharmaceutical composition comprises immunoglobulin capable of binding specifically to one or more bacterial lipopolysaccharides, wherein the immunoglobulin comprises, consists essentially of, or consists of immunoglobulin from colostrum.

41. A pharmaceutical composition for reducing the amount of one or more lipopolysaccharides present in a subject or a sample from a subject, wherein the pharmaceutical composition comprises a multimerizing reagent capable of binding to one or more lipopolysaccharides from gram-negative bacteria, wherein when bound to and/or complexed with said one or more lipopolysaccharides present in the subject said multimerizing reagent is capable of further multimerization.

42. Use of immunoglobulin capable of binding to one or more lipopolysaccharides from gram-negative bacteria in the preparation of a medicament for use in treating or preventing a bacterial infection of gram-negative bacteria or of a disease or condition caused by or associated with a bacterial infection of gram-negative bacteria, wherein the immunoglobulin comprises, consists essentially of, or consists of immunoglobulin from colostrum.

43. Use of a multimerizing reagent capable of binding to one or more lipopolysaccharides from gram-negative bacteria in the preparation of a medicament for use in treating or preventing a bacterial infection of gram-negative bacteria or of a disease or condition caused by or associated with a bacterial infection of gram-negative bacteria, wherein when bound to and/or complexed with said one or more lipopolysaccharides present in the subject said multimerizing reagent is capable of further multimerization.

44. A method of treating or preventing a disease or condition caused by or associated with a bacterial infection in a subject in need thereof, the method comprising administering to the subject an effective amount of an antibody-binding agent, wherein the antibody-binding reagent is capable of specifically binding one or more endogenous lipopolysaccharide-binding antibodies present in the subject.

45. The method according to claim 44, wherein the antibody-binding agent is an antibody or antibody fragment, an antibody binding protein or fragment thereof, or a combination thereof.

46. The method according to claim 45 wherein the antibody-binding agent is selected from the group consisting of: an anti-Ig antibody or a fragment thereof, Protein A or an antibody-binding fragment thereof, Protein G or an antibody-binding fragment thereof, an anti IgA antibody or an IgA antibody-binding fragment thereof, or an IgA-binding reagent.

47. The method according to claim 44, wherein the antibody-binding agent is Concanavalin A.

48. A method of treating or preventing a disease or condition caused by or associated with a bacterial infection in a subject in need thereof, or treating or preventing a bacterial infection in a subject in need thereof, the method comprising administering to the subject an effective amount of Concanavalin A or an analogue, derivative, or synthetic form thereof.

49. The method according to any one of the preceding claims, wherein the method comprises administering to the subject an additional therapeutic agent.

50. The method according to claim 49, wherein the additional therapeutic agent is an antibiotic selected from the group consisting of aminoglycosides, such as gentamicin, amikacin; beta-lactams; carbapenems, including imipenem, meropenem; cephalosporins, including cefotaxime, ceftazidime; chloramphenicols; fluorquinolones, such as ciprofloxacin, delafloxacin; fosfomycin; penicillins; polymyxins, such as colistin, polymyxin B; glycylcycline, such as tigecycline; sulphonamides, such as co-trimoxazole; tetracyclines, including doxycycline, eravacycline, minocycline, omadacycline; and ureidopenicillins, such as piperacillin; and any combination of two or more thereof, including combinations such as ceftolozane/tazobactam, ceftazidime/avibactam, meropenem/vaborbactam, and imipenem/cilastatin/relebactam.
